(19) United States
(12) Patent Application Publication
(10) Pub. No.: US 2024/0016483 A1
(43) Pub. Date: Jan. 18, 2024

(54) OPTICALLY ENHANCED INSTRUMENT WITH LASER FLUORESCING CAPABILITIES

(71) Applicant: GYRUS ACMI, INC. D/B/A OLYMPUS SURGICAL TECHNOLOGIES AMERICA, Westborough, MA (US)

(72) Inventors: Nikhil M. Murdeshwar, Maple Grove, MA (US); Thomas J. Holman, Princeton, MA (US)

(21) Appl. No.: 18/032,114

(22) PCT Filed: Oct. 15, 2021
(86) PCT No.: PCT/US2021/055193

§ 371 Date: Apr. 14, 2023

Related U.S. Application Data
(60) Provisional application No. 63/092,664, filed on Oct. 16, 2020.
(60) Provisional application No. 63/127,483, filed on Dec. 18, 2020.
(60) Provisional application No. 63/213,853, filed on Jun. 23, 2021.

(51) Int. Cl. A61B 10/04; A61B 10/06; A61B 1/04; A61B 1/06; A61B 1/018

(52) U.S. Cl. CPC A61B 10/04; A61B 10/06; A61B 1/043; A61B 1/0638; A61B 1/018

(57) ABSTRACT

A surgical instrument comprises an elongate body comprising a proximal end portion and a distal end portion and a tissue separator coupled to the distal end portion that is configured to engage sample tissue for collection, wherein the tissue separator is at least partially made from a material that allows light to pass through the tissue separator. A method of collecting biological matter using a tissue retrieval device comprises inserting the tissue retrieval device into anatomy of a patient, guiding a tissue collector of the tissue retrieval device to a target tissue, viewing the target tissue through the tissue collector and collecting biological matter from the target tissue with the tissue retrieval device.

### PRIORITY CLAIM

This application claims the benefit of priority to U.S. Provisional Patent Application Ser. No. 63/092,664, filed Oct. 16, 2020, U.S. Provisional Patent Application Ser. No. 63/127,483 filed on Dec. 18, 2020, and U.S. Provisional Patent Application Ser. No. 63/213,853 filed Jun. 23, 2021, the contents of which are hereby incorporated by reference in their entireties.

### TECHNICAL FIELD

The present disclosure relates generally to medical devices comprising elongate bodies configured to be inserted into incisions or openings in anatomy of a patient to provide diagnostic or treatment operations.

More specifically, the present disclosure relates to medical devices that can be inserted into anatomy of a patient to perform a biological matter removal process, such as by cutting sample tissue for analysis.

### BACKGROUND

Endoscopes can be used for one or more of 1) providing passage of other devices, e.g., therapeutic devices or tissue collection devices, toward various anatomical portions, and 2) imaging of such anatomical portions. Such anatomical portions can include gastrointestinal tract (e.g., esophagus, stomach, duodenum, pancreaticobiliary duct, intestines, colon, and the like), renal area (e.g., kidney(s), ureter, bladder, urethra) and other internal organs (e.g., reproductive systems, sinus cavities, submucosal regions, respiratory tract), and the like.

Conventional endoscopes can be involved in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices for sampling or treating an anatomical region, and providing suction passageways for collecting fluids (e.g., saline or other preparations) and the like.

In conventional endoscopy, the distal portion of the endoscope can be configured for supporting and orienting a therapeutic device, such as with the use of an elevator. In some systems, two endoscopes can be configured to work together with a first endoscope guiding a second endoscope inserted therein with the aid of the elevator. Such systems can be helpful in guiding endoscopes to anatomic locations within the body that are difficult to reach. For example, some anatomic locations can only be accessed with an endoscope after insertion through a circuitous path.

### SUMMARY

The present disclosure recognizes that problems to be solved with conventional medical devices, and in particular endoscopes and duodenoscopes used to retrieve sample biological matter, include, among other things, 1) the difficulty in navigating endoscopes, and instruments inserted therein, to locations in anatomical regions deep within a patient, 2) the disadvantage of only being able to obtain small tissue sample sizes 3) the increased time and associated cost of having to repeatedly remove and reinsert medical devices to obtain a sufficient quantity of sample material, and 4), the difficulty of incorporating features (e.g., steerability and tissue collection features) into small-diameter devices, particularly without obstructing optical devices (e.g., imaging and lighting components) mounted to the endoscope. Such problems can be particularly present in duodenoscopy procedures (e.g., Endoscopic Retrograde Cholangio-Pancreatography, hereinafter “ERCP” procedures) where an auxiliary scope (also referred to as daughter scope, or cholangioscope) can be attached and advanced through the working channel of a “main scope” (also referred to as mother scope or duodenoscope). Furthermore, tissue collection and retrieval devices used to remove the sample matter can be inserted through the auxiliary scope. As such, the duodenoscope, auxiliary scope and tissue retrieval device become progressively smaller and more difficult to maneuver and perform interventions and treatments.

The present disclosure can help provide solutions to these and other problems by providing systems, devices and methods relating to inserting tissue retrieval devices, such as biopsy forceps, with an auxiliary scope having a small-diameter passage. The tissue retrieval devices can be tethered or otherwise attached to a distal end of an endoscope to allow the tissue retrieval device to be sized beyond the constraints of the lumen of the endoscope. The tissue retrieval device can thereby have increased capacity for storing obtained sample tissue, thereby reducing, or eliminating the need to remove the endoscope to empty the tissue retrieval device for another sample collection insertion iteration.

Furthermore, in order to facilitate navigation of the endoscope with the tissue retrieval device located distally thereof and the tissue collection process, the tissue retrieval device can be optically enhanced, such as by being made of translucent or clear materials to allow visibility of optical devices through and into the tissue retrieval device. Other optically enhanced materials can include reflective materials to allow for interaction of the material with light to improve recognition by the optical device. Optically enhanced tissue retrieval devices can be configured to bend light waves, such as to provide optical magnification. The optically enhanced material can allow for viewing of: 1) target tissue to be collected by the tissue retrieval device, 2) tissue inside the tissue retrieval device, 3) newly exposed tissue after some target tissue has been separated from the anatomy, and 4) components of the tissue retrieval device relative to the target tissue, as well as other benefits.

Additionally, owing in part to being freed of the size constraints of the endoscope lumen, the tissue retrieval device can include features to facilitate obtaining multiple samples of tissue without previously collected samples becoming dislodged from (e.g., falling out of) the tissue retrieval device and to increase the holding capacity of the tissue retrieval device. Thus, the tissue retrieval devices can be configured to hold one or more pieces of sample material, thereby allowing collection of multiple samples and larger samples in a single insertion pass.

As such, the present disclosure can help solve the problems referenced above and other problems by 1) reducing the number of times a tissue retrieval device needs to be inserted and reinserted into the anatomy, and 2) increasing the capacity of sample material collected with each insertion, among other things, as is described herein, such as by locating distally of an endoscope a tissue retrieval device that can be larger than the lumen of the endoscope to increase size and that can be optically enhanced to reduce or eliminate interference with imaging capabilities.

The present disclosure also recognizes that problems to be solved in performing medical procedures include the ability to properly identify target tissue for removal. For example, ductal malignancies can include endometriosis and cancerous or pre-cancerous material, including carcinoma, sarcoma, myeloma, leukemia, lymphoma and mixed types of cancers.

Treatment for these ductal malignancies can involve removing the diseased tissue either as an end in itself or to perform a biopsy to determine a next course of action. As such, it is desirable to identify the ductal malignancies such that other healthy tissue in the duct or abdomen is not unnecessarily removed and to prevent having to go back into the patient in a follow-up procedure to remove additional tissue. For example, it is desirable to remove all of the extra-uterine endometrium tissue to treat the disease and it is desirable to collect a volume of tissue sufficient to perform a biopsy.

Identification of endometrium tissue and cancerous tissue can be facilitated by the use of dyes whereby a patient ingests a dye that can metabolize to or otherwise be absorbed by the endometrium and cancerous tissue. The dye can then be energized with light of a particular wavelength to illuminate the tissue containing the dye. However, use of dyes requires light be introduced into the surgical site, which typically requires use of an additional instrument. Furthermore, it can be difficult to direct the energization light onto the target tissue while operating a device to engage the tissue due to, for example, obstruction of the device itself of obstruction of other instruments working with the device.

The present subject matter can provide solutions to this problem and other problems, such as by providing systems incorporating light emitters into a surgical instrument in such a way that a surgical tool portion (e.g., a tissue collector or separator) of the surgical instrument can be directly illuminated via emitted light, without the need for an additional or separate tool. Furthermore, tissue retrieval devices can be made of transparent tissue separators and collectors, e.g., blades and jaws, to allow energization light to pass through the tissue collector and energize the dye. The light can be provided in different wavelengths to provide different energization to tissue-illuminating dyes. Methods of performing surgical procedures with such systems are also described herein.

The terms “tissue retrieval device” and “biopsy instrument” are used throughout the present disclosure, however a tissue retrieval device or biopsy instrument can alternatively or additionally comprise a biological matter collection device, a biological matter retrieval device, a tissue collection device and tissue retrieval device.

In an example, a tissue separation device can comprise an elongate body comprising a proximal end portion and a distal end portion, and a tissue separator coupled to the distal end portion, the tissue separator configured to engage sample tissue for retrieval, wherein the tissue separator is at least partially made from a material that allows light to pass through the tissue separator

In another example, a method of collecting biological matter using a tissue retrieval device can comprise inserting the tissue retrieval device into anatomy of a patient, guiding a tissue collector of the tissue retrieval device to a target tissue area, viewing the target tissue through the tissue collector, and collecting biological matter from the target tissue with the tissue retrieval device.

In an additional example, a surgical instrument can comprise an endoscope and a tissue retrieval device. The endoscope can comprise an insertion shaft extending from a proximal end to a distal end, a working channel extending at least partially through the insertion shaft, and an imaging system coupled to the insertion shaft, the imaging system having a field of view projecting distally of the working channel. The tissue retrieval device can comprise an elongate shaft positionable in the working channel, the elongate shaft extending along an axis and configured for insertion into anatomy, and a tissue collection device coupled to the elongate shaft and configured to separate tissue from the anatomy, wherein the tissue collection device is optically enhanced to interact with the field of view of the imaging system.

In an example, a biopsy instrument can comprise a tissue separator device and an elongate control element. The tissue separator device can comprise a base, a tissue separator mounted to the base, and an actuation mechanism coupled to the tissue separator. The elongate control element can be coupled to the actuation mechanism to manipulate the tissue separator. The tissue separator can be at least partially made of a material capable of transmitting light.

In another example, a method of collecting biological matter using a biopsy instrument comprises tethering the biopsy instrument to a distal end portion of an endoscope, inserting the endoscope with the biopsy instrument into anatomy of a patient, guiding the biopsy instrument to a target tissue site, viewing the target tissue through the biopsy instrument, and collecting biological matter from the target tissue site with the biopsy instrument.

In an example, a surgical instrument can comprise a scope and a tissue retrieval device. The scope can comprise an elongate body extending from a proximal end portion to a distal end portion, a working channel extending at least partially through the elongate body, an imaging component coupled to the elongate body and having a field of view projecting distally of the working channel and a first light emitter configured to project light of a first wavelength from the elongate body suitable for energizing a light-emitting material. The tissue retrieval device can comprise an elongate shaft positionable in the working channel to extend along an axis and configured for insertion into anatomy and a tissue collection device coupled to the elongate shaft and configured to separate tissue from the anatomy.

In another example, a method of collecting biological matter using a tissue retrieval device can comprise inserting the tissue retrieval device into anatomy of a patient, guiding a tissue collector of the tissue retrieval device to a target tissue, illuminating the target tissue with an energization light and collecting biological matter from the target tissue with the tissue collector.

### BRIEF DESCRIPTION OF DRAWINGS

FIG. 1 is a schematic diagram of an endoscopy system comprising an imaging and control system and an endoscope, such as duodenoscope, with which the biological matter collection systems and devices of the present disclosure can be used.

FIG. 2 is a schematic diagram of the imaging and control system of FIG. 1 showing the imaging and control system connected to the endoscope.

FIG. 3A is a schematic top view of a distal portion of the endoscope of FIG. 2 comprising a camera module including optical components for a side-viewing endoscope and an elevator mechanism.

FIG. 3B is an enlarged cross-sectional view taken along the plane 3B-3B of FIG. 3A showing the optical components.

FIG. 3C is an enlarged cross-sectional view taken along the plane 3C-3C of FIG. 3A showing the elevator mechanism.

FIG. 4 is a schematic illustration of a distal portion of an endoscope being used to position an auxiliary scope proximate a duodenum, the auxiliary scope being configured to receive a tissue retrieval device, including a tethered biopsy instrument, of the present disclosure.

FIG. 5A is a schematic illustration of a tissue retrieval device of the present disclosure comprising an elongate shaft and a translucent tissue collector.

FIG. 5B is a close-up view of a distal end of the tissue retrieval device of FIG. 5A showing the translucent tissue collector disposed within an auxiliary endoscope.

FIG. 6A is a schematic illustration of a translucent tissue collector comprising forceps in a closed state.

FIG. 6B is a schematic illustration of the translucent tissue collector of FIG. 6A with the forceps in an open state.

FIG. 7A is a schematic illustration of a translucent tissue collector comprising a boring device extending from an endoscope having an imaging device.

FIG. 7B is a schematic cross-sectional illustration of the translucent tissue collector of FIG. 7A with collected tissue inside the translucent tissue collector.

FIG. 8A is a schematic illustration of an endoscopy system comprising an endoscope and a tethered biopsy instrument.

FIG. 8B is a side view of a forceps suitable for use as the biopsy instrument of FIG. 8A.

FIG. 9 is a schematic illustration of a biopsy instrument comprising forceps having a tissue retention system comprising a sponge and needles.

FIG. 10 is schematic illustration of a biopsy instrument comprising forceps having expandable jaws.

FIG. 11 is a schematic illustration of a biopsy instrument comprising forceps having a flexible jaw.

FIG. 12 is a block diagram illustrating methods of collecting biological matter from a patient using tethered biopsy instruments of the present disclosure.

FIG. 13 is a schematic illustration of a tissue collection instrument comprising forceps having a light emitter configured to energize light-emitting material.

FIG. 14 is a block diagram illustrating methods of illuminating tissue having a light-emitting dye with a tissue collection device having built-in energization capabilities and an optically-enhanced tissue separator.

FIG. 15 is block diagram illustrating methods of collecting biological matter from a patient using the tissue retrieval devices of the present disclosure.

### DETAILED DESCRIPTION

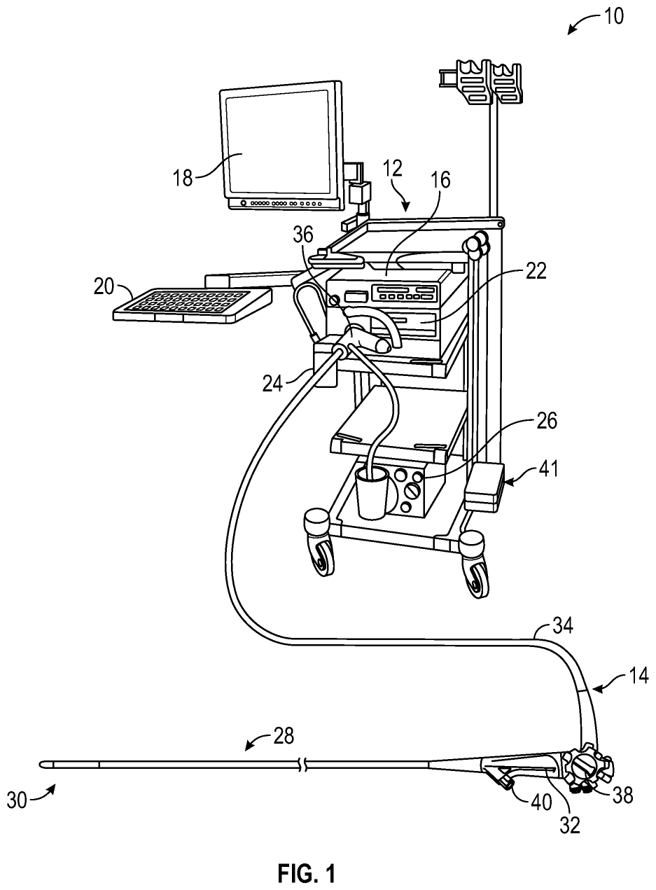

FIG. 1 is a schematic diagram of endoscopy system 10 comprising imaging and control system 12 and endoscope 14. The system of FIG. 1 is an illustrative example of an endoscopy system suitable for use with the systems, devices and methods described herein, such as tethered and optically enhanced biological matter and tissue collection, retrieval and storage devices and biopsy instruments that can be used for obtaining samples of tissue or other biological matter to be removed from a patient for analysis or treatment of the patient. According to some examples, endoscope 14 can be insertable into an anatomical region for imaging and/or to provide passage of or attachment to (e.g., via tethering) one or more sampling devices for biopsies, or one or more therapeutic devices for treatment of a disease state associated with the anatomical region. Endoscope 14 can, in advantageous aspects, interface with and connect to imaging and control system 12. In the illustrated example, endoscope 14 comprises a duodenoscope, though other types of endoscopes can be used with the features and teachings of the present disclosure.

Imaging and control system 12 can comprise control unit 16, output unit 18, input unit 20, light source 22, fluid source 24 and suction pump 26.

Imaging and control system 12 can include various ports for coupling with endoscopy system 10. For example, control unit 16 can include a data input/output port for receiving data from and communicating data to endoscope 14. Light source 22 can include an output port for transmitting light to endoscope 14, such as via a fiber optic link. Fluid source 24 can include a port for transmitting fluid to endoscope 14. Fluid source 24 can comprise a pump and a tank of fluid or can be connected to an external tank, vessel or storage unit. Suction pump 26 can comprise a port used to draw a vacuum from endoscope 14 to generate suction, such as for withdrawing fluid from the anatomical region into which endoscope 14 is inserted. Output unit 18 and input unit 20 can be used by an operator of endoscopy system 10 to control functions of endoscopy system 10 and view output of endoscope 14. Control unit 16 can additionally be used to generate signals or other outputs from treating the anatomical region into which endoscope 14 is inserted. In examples, control unit 16 can generate electrical output, acoustic output, a fluid output and the like for treating the anatomical region with, for example, cauterizing, cutting, freezing and the like.

Endoscope 14 can comprise insertion section 28, functional section 30 and handle section 32, which can be coupled to cable section 34 and coupler section 36.

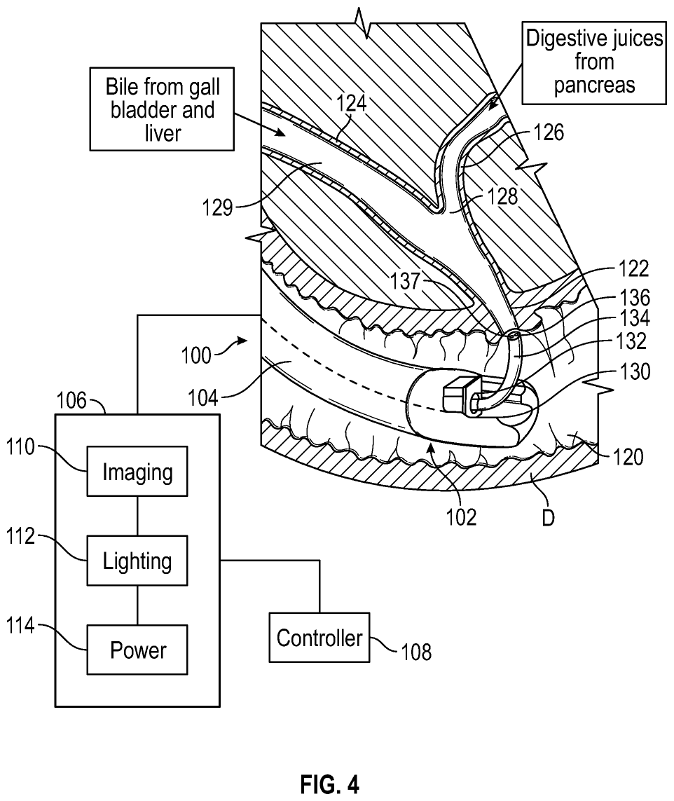

Insertion section 28 can extend distally from handle section 32 and cable section 34 can extend proximally from handle section 32. Insertion section 28 can be elongate and include a bending section, and a distal end to which functional section 30 can be attached. The bending section can be controllable (e.g., by control knob 38 on handle section 32) to maneuver the distal end through tortuous anatomical passageways (e.g., stomach, duodenum, kidney, ureter, etc.). Insertion section 28 can also include one or more working channels (e.g., an internal lumen) that can be elongate and support insertion of one or more therapeutic tools of functional section 30, such as auxiliary scope 134 of FIG. 4. The working channel can extend between handle section 32 and functional section 30. Additional functionalities, such as fluid passages, guide wires, and pull wires can also be provided by insertion section 28 (e.g., via suction or irrigation passageways, and the like).

Handle section 32 can comprise knob 38 as well as ports 40. Knob 38 can be coupled to a pull wire, or other actuation mechanisms, extending through insertion section 28. Ports 40 can be configured to couple various electrical cables, guide wires, auxiliary scopes, tissue collection devices of the present disclosure, fluid tubes and the like to handle section 32 for coupling with insertion section 28.

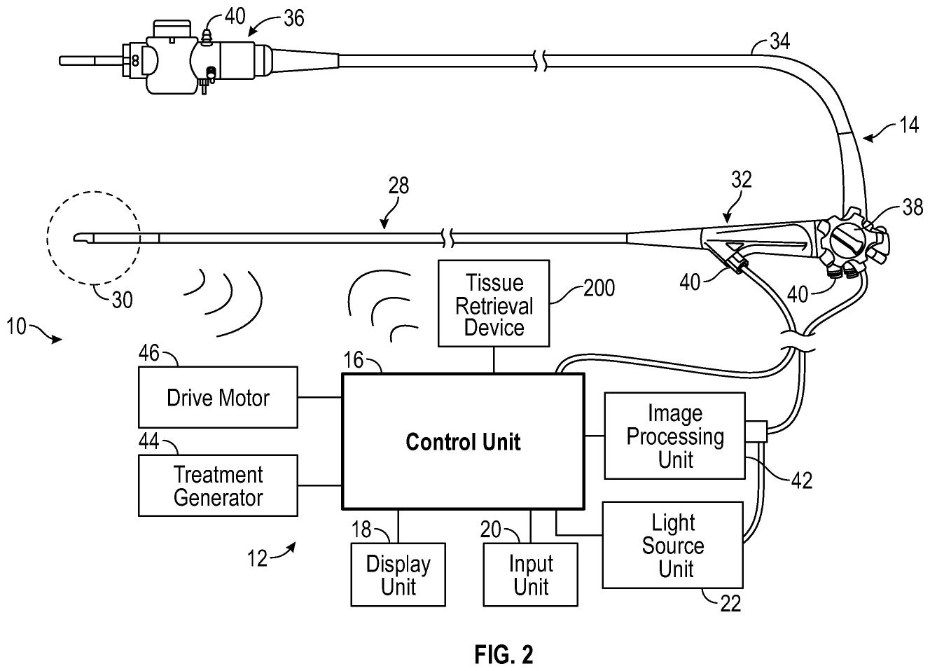

Imaging and control system 12, according to examples, can be provided on a mobile platform (e.g., cart 41) with shelves for housing light source 22, suction pump 26, image processing unit 42 (FIG. 2), etc. Alternatively, several components of imaging and control system 12 shown in FIGS. 1 and 2 can be provided directly on endoscope 14 so as to make the endoscope “self-contained.”

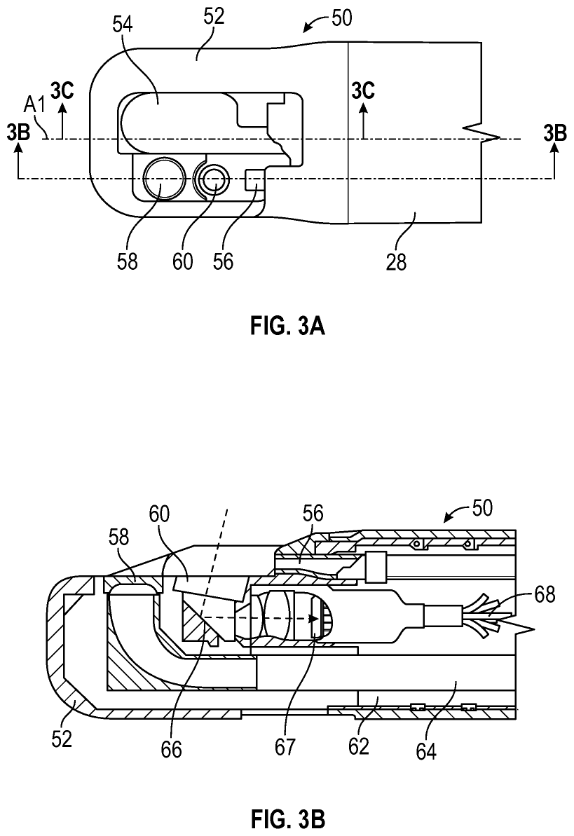

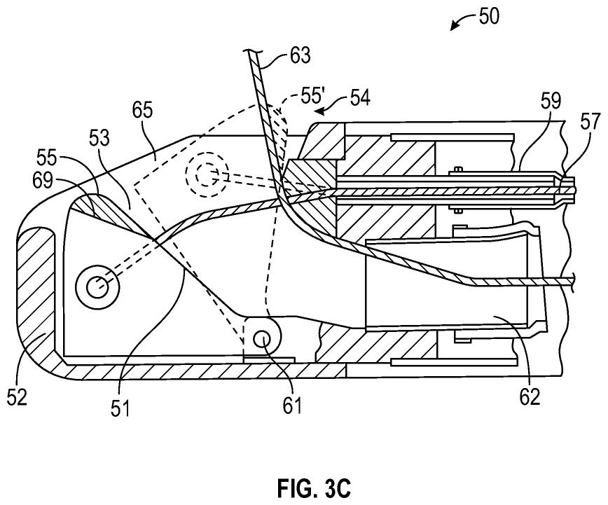

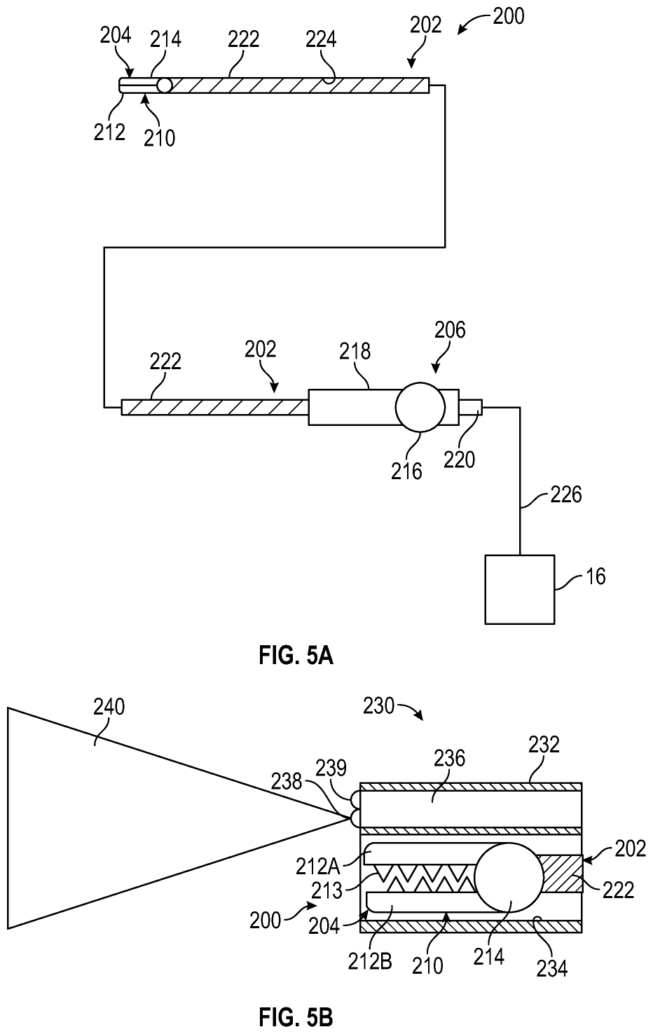

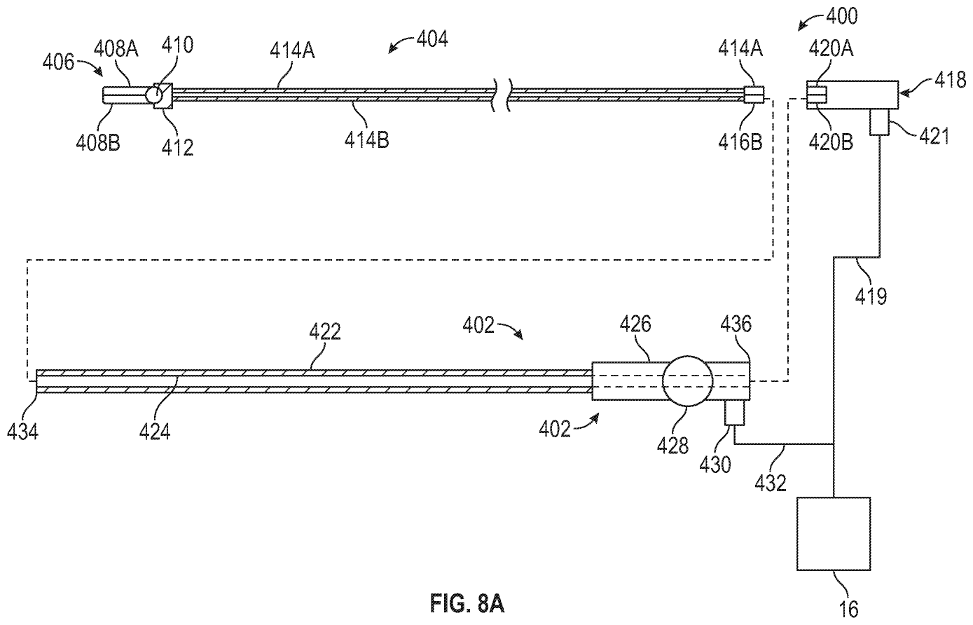

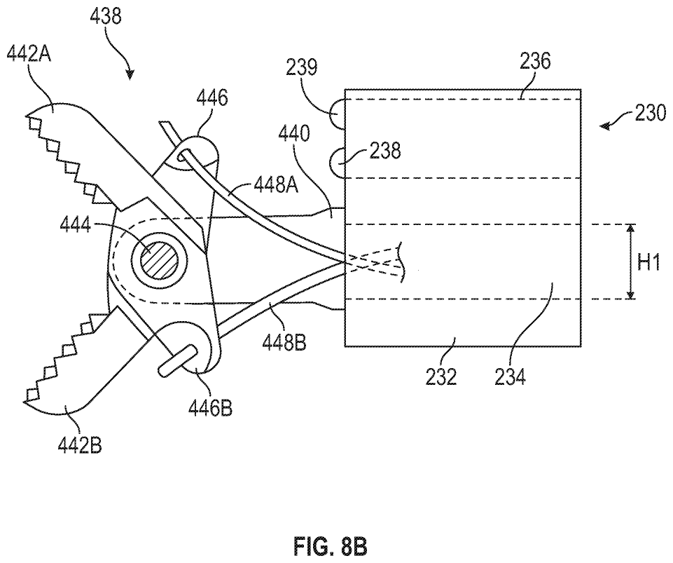

Functional section 30 can comprise components for treating and diagnosing anatomy of a patient. Functional section 30 can comprise an imaging device, an illumination device and an elevator, such as is described further with reference to elevator 54 of FIGS. 3A-3C. Functional section 30 can further comprise optically enhanced biological matter and tissue collection and retrieval devices as are described herein. For example, functional section 30 can comprise one or more electrodes conductively connected to handle section 32 and functionally connected to imaging and control system 12 to analyze biological matter in contact with the electrodes based on comparative biological data stored in imaging and control system 12. In other examples, functional section 30 can directly incorporate tissue collectors similar to the tissue retrieval devices described with reference to FIGS. 5A-7B and the biopsy devices described with reference to FIGS. 8A-11.

FIG. 2 is a schematic diagram of endoscopy system 10 of FIG. 1 comprising imaging and control system 12 and endoscope 14. FIG. 2 schematically illustrates components of imaging and control system 12 coupled to endoscope 14, which in the illustrated example comprises a duodenoscope. Imaging and control system 12 can comprise control unit 16, which can include or be coupled to image processing unit 42, treatment generator 44 and drive unit 46, as well as light source 22, input unit 20 and output unit 18. As is discussed below in greater detail with reference to FIGS. 4-5B, control unit 16 can comprise, or can be in communication with, endoscope 100, surgical instrument 200 and endoscopy system 400, which can comprise a device configured to engage tissue and collect and store a portion of that tissue and through which imaging equipment (e.g., a camera) can view target tissue via inclusion of optically enhanced materials and components. Control unit 16 can be configured to activate a camera to view target tissue distal of surgical instrument 200 and endoscopy system 400, which can be fabricated of a translucent material to minimize the impacts of the camera being obstructed or partially obstructed by the tissue retrieval device. Likewise, control unit 16 can be configured to activate light source 22 to shine light on surgical instrument 200, which can include select components that are configured to reflect light in a particular manner, such as tissue cutters being enhanced with reflective particles.

Image processing unit 42 and light source 22 can each interface with endoscope 14 (e.g., at functional unit 30) by wired or wireless electrical connections. Imaging and control system 12 can accordingly illuminate an anatomical region, collect signals representing the anatomical region, process signals representing the anatomical region, and display images representing the anatomical region on display unit 18. Imaging and control system 12 can include light source 22 to illuminate the anatomical region using light of desired spectrum (e.g., broadband white light, narrow-band imaging using preferred electromagnetic wavelengths, and the like). Imaging and control system 12 can connect (e.g., via an endoscope connector) to endoscope 14 for signal transmission (e.g., light output from light source, video signals from imaging system in the distal end, diagnostic and sensor signals from a diagnostic device, and the like).

Fluid source 24 (FIG. 1) can be in communication with control unit 16 and can comprise one or more sources of air, saline or other fluids, as well as associated fluid pathways (e.g., air channels, irrigation channels, suction channels) and connectors (barb fittings, fluid seals, valves and the like). Fluid source 24 can be utilized as an activation energy for a biasing device or a pressure-applying device of the present disclosure. Imaging and control system 12 can also include drive unit 46, which can be an optional component. Drive unit 46 can comprise a motorized drive for advancing a distal section of endoscope 14, as described in at least PCT Pub. No. WO 2011/140118 A1 to Frassica et al., titled “Rotate-to-Advance Catheterization System,” which is hereby incorporated in its entirety by this reference.

FIGS. 3A-3C illustrate a first example of functional section 30 of endoscope 14 of FIG. 2. FIG. 3A illustrates a top view of functional section 30. FIG. 3B illustrates a cross-sectional view of functional section 30 taken along section plane 3B-3B of FIG. 3A. FIG. 3C illustrates a cross-sectional view of functional section 30 taken along section plane 3C-3C of FIG. 3A. FIGS. 3A-3C illustrate “side-viewing endoscope” (e.g., duodenoscope) camera module 50. In side-viewing endoscope camera module 50, illumination and imaging systems are positioned such that the viewing angle of the imaging system corresponds to a target anatomy lateral to central longitudinal axis A1 of endoscope 14. However, the biological matter retrieval devices can be used with other types of endoscopes, such as “end-viewing endoscopes.”

In the example of FIGS. 3A and 3B, side-viewing endoscope camera module 50 can comprise housing 52, elevator 54, fluid outlet 56, illumination lens 58 and objective lens 60. Housing 52 can form a fluid tight coupling with insertion section 28. Housing 52 can comprise opening for elevator 54. Elevator 54 can comprise a mechanism for moving a device inserted through insertion section 28, such as auxiliary scope 134 of FIG. 4. In particular, elevator 54 can comprise a device that can bend an elongate device extended through insertion section 28 along axis A1, as is discussed in greater detail with reference to FIG. 3C. Elevator 54 can be used to bend the elongate device at an angle to axis A1 to thereby treat or access the anatomical region adjacent side-viewing endoscope camera module 50. Elevator 54 is located alongside, e.g., radially outward of axis A1, illumination lens 58 and objective lens 60.

As can be seen in FIG. 3B, insertion section 28 can comprise central lumen 62 through which various components (e.g., auxiliary scope 134 (FIG. 4) can be extended to connect functional section 30 with handle section 32 (FIG. 2). For example, illumination lens 58 can be connected to light transmitter 64, which can comprise a fiber optic cable or cable bundle extending to light source 22 (FIG. 1). Likewise, objective lens 60 can be coupled to prism 66 and imaging unit 67, which can be coupled to wiring 68. Also, fluid outlet 56 can be coupled to fluid line 69, which can comprise a tube extending to fluid source 24 (FIG. 1). Other elongate elements, e.g., tubes, wires, cables, can extend through lumen 62 to connect functional section 30 with components of endoscopy system 10, such as suction pump 26 (FIG. 1) and treatment generator 44 (FIG. 2).

FIG. 3C a schematic cross-sectional view taken along section plane 3C-3C of FIG. 30 showing elevator 54. Elevator 54 can comprise deflector 55 that can be disposed in space 53 of housing 52. Deflector 55 can be connected to wire 57, which can extend through tube 59 to connect to handle section 32. Wire 57 can be actuated, such as by rotating a knob, pulling a lever, or pushing a button on handle section 32. Movement of wire 57 can cause rotation, e.g., clockwise, from a first position of deflector 55 about pin 61 to a second position of deflector 55, indicated by 55′. Deflector 55 can be actuated by wire 57 to move the distal portion of instrument 63 extending through window 65 in housing 52.

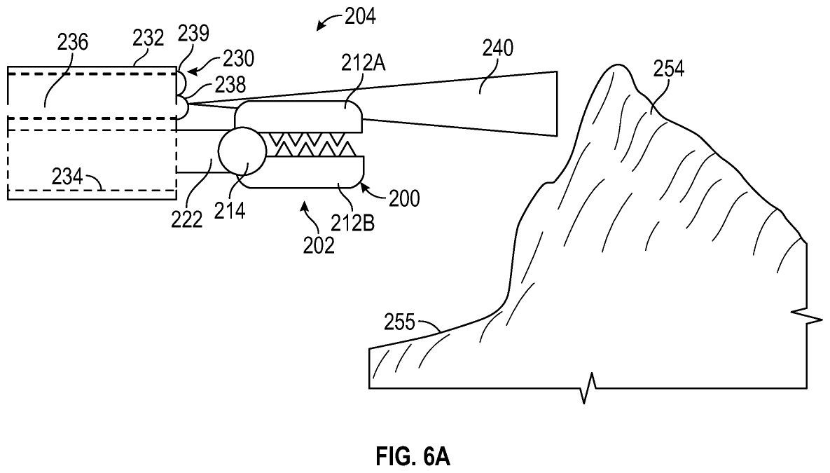

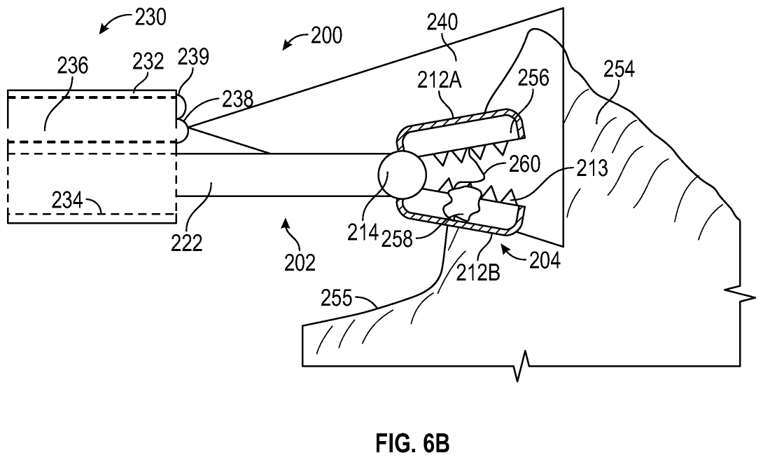

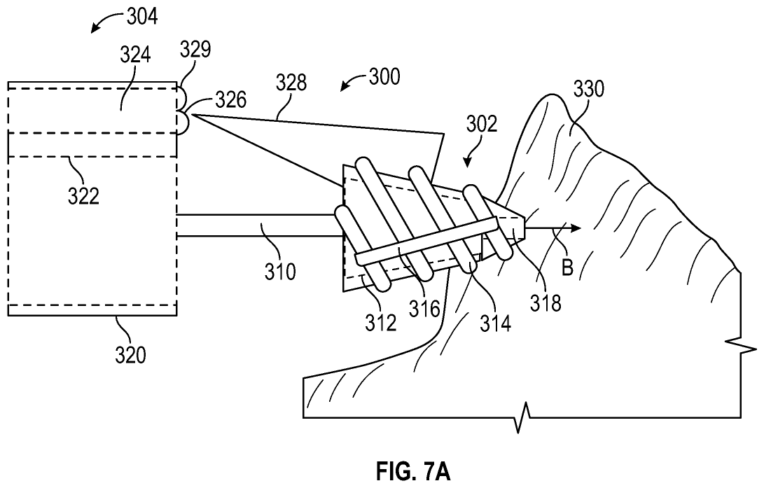

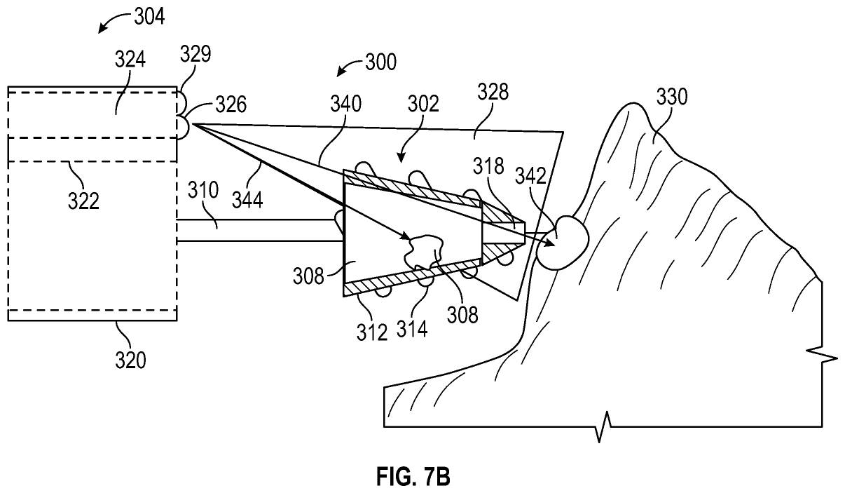

Housing 52 can comprise accommodation space 53 that houses deflector 55. Instrument 63 can comprise forceps, a guide wire, a catheter, or the like that extends through lumen 62. Instrument 63 can additionally comprise auxiliary scope 134 of FIG. 4, or a tissue collection device such as surgical instrument 200 of FIGS. 5A-6B and tissue retrieval device 300 (FIG. 7A), as well as other instruments such as biopsy instruments 404 of FIG. 8A. A proximal end of deflector 55 can be attached to housing 62 at pin 61 provided to the rigid tip 21. A distal end of deflector 55 can be located below window 65 within housing 62 when deflector 55 is in the lowered, or un-actuated, state. The distal end of deflector 55 can at least partially extend out of window 65 when deflector 55 is raised, or actuated, by wire 57. Instrument 63 can slide on angled ramp surface 51 of deflector 55 to initially deflect the distal end of instrument 63 toward window 65. Angled ramp surface 51 can facilitate extension of the distal portion of instrument 63 extending from window 65 at a first angle relative to the axis of lumen 62. Angled ramp surface 51 can include groove 69, e.g., a v-notch, to receive and guide instrument 63. Deflector 55 can be actuated to bend instrument 63 at a second angle relative to the axis of lumen 62, which is closer to perpendicular that the first angle. When wire 57 is released, deflector 55 can be rotated, e.g., counter-clockwise, back to the lowered position, either by pushing or relaxing of wire 57. In examples, instrument 63 can comprise a cholangioscope or auxiliary scope 134 (FIG. 4).

Side-viewing endoscope camera module 50 of FIGS. 3A-3C can include optical components (e.g., objective lens 60, prism 66, imaging unit 67, wiring 68) for collection of image signals, lighting components (e.g., illumination lens 58, light transmitter 64) for transmission or generation of light. Endoscope camera module 50 can also include a photosensitive element, such as a charge-coupled device (“CCD” sensor) or a complementary metal-oxide semiconductor (“CMOS”) sensor. In either example, imaging unit 67 can be coupled (e.g., via wired or wireless connections) to image processing unit 42 (FIG. 2) to transmit signals from the photosensitive element representing images (e.g., video signals) to image processing unit 42, in turn to be displayed on a display such as output unit 18. In various examples, imaging and control system 12 and image processing unit 67 can be configured to provide outputs at desired resolution (e.g., at least 480p, at least 720p, at least 1080p, at least 4K UHD, etc.) suitable for endoscopy procedures.

Thus, as endoscope 100 is inserted further into the anatomy, the complexity with which it must be maneuvered and contorted increases, as described with reference to FIG. 4. Furthermore, in order to reach locations even further in the anatomy, additional devices can be used, e.g., instrument 63 in the form of auxiliary scope 134. As such, the cross-sectional area, e.g., diameter, of subsequently nested devices becomes smaller, thereby requiring even smaller devices that can be difficult to manufacture and manipulate, or satisfactorily produce results without repeated interventions (e.g., interactions with the patient), as is described with reference to FIGS. 5A-7B.

FIG. 4 is a schematic illustration of distal portion of endoscope 100 according to the present disclosure positioned in duodenum D. Endoscope 100 can comprise functional module 102, insertion section module 104, and control module 106. Control module 106 can include controller 108. Control module 106 can include other components, such as those described with reference to endoscopy system 10 (FIG. 1) and control unit 16 (FIG. 2). Additionally, control module 106 can comprise components for controlling a camera and a light source connected to auxiliary scope 134, such as imaging unit 110, lighting unit 112 and power unit 114. Endoscope 100 can be configured similarly as endoscope 14 of FIGS. 1 and 2.

Duodenum D can comprise duct wall 120, sphincter of Oddi 122, common bile duct 124 and main pancreatic duct 126. Duodenum D comprises an upper part of the small intestine. Common bile duct 124 carries bile from the gallbladder and liver (not illustrated) and empties the bile into the duodenum D through sphincter of Oddi 122. Main pancreatic duct 126 carries pancreatic juice from the exocrine pancreas (not illustrated) to common bile duct 124. Sometimes it can be desirable to remove biological matter, e.g., tissue, from bile duct 124 or pancreatic duct 126 to analyze the tissue to, for example, diagnose diseases or maladies of the patient such as cancer.

Functional module 102 can comprise elevator portion 130. Endoscope 100 can further comprise lumen 132 and auxiliary scope 134. Auxiliary scope 134 can comprise lumen 136. Auxiliary scope 134 can itself include functional components, such as camera lens 137 and a light lens (not illustrated) coupled to control module 106, to facilitate navigation of auxiliary scope 134 from endoscope 100 through the anatomy and to facilitate viewing of components extending from lumen 132.

In certain duodenoscopy procedures (e.g., Endoscopic Retrograde Cholangio-Pancreatography, hereinafter “ERCP” procedures) an auxiliary scope (also referred to as daughter scope, or cholangioscope), such as auxiliary scope 134, can be attached and advanced through lumen 132 (or central lumen 62 of insertion section 28 of endoscope 14 in FIG. 3B) of the “main scope” (also referred to as mother scope, or duodenoscope), such as endoscope 100. As discussed in greater detail below, auxiliary scope 134 can be guided into sphincter of Oddi 122. Therefrom, a surgeon operating auxiliary scope 134 can navigate auxiliary scope 134 through lumen 132 toward the gall bladder, liver or other locations in the gastrointestinal system to perform various procedures. The surgeon can navigate auxiliary scope 134 past entry 128 of main pancreatic duct 126 and into passage 129 of common bile duct 124, or into entry 128. Auxiliary scope 134 can be used to guide an additional device to the anatomy to obtain biological matter, such as by passage through or attachment to lumen 136. The additional device can have its own functional devices, such as a light source, camera, tissue separators, accessories, and biopsy channel, for therapeutic procedures. As described with reference to FIGS. 5A-7B, the additional device can include various features, such as forceps or an auger, for gathering biological matter, such as tissue. As described with reference to FIGS. 8A-11, the additional device can comprise a biopsy device tethered to the endoscope and that has tissue collection capacity enhancement features. The biological matter can then be removed from the patient, typically by removal of the additional device from the auxiliary device, so that the removed biological matter can be analyzed to diagnose one or more conditions of the patient. According to several examples, endoscope 100 can be suitable for the removal of cancerous or pre-cancerous matter (e.g., carcinoma, sarcoma, myeloma, leukemia, lymphoma and the like), endometriosis evaluation, biliary ductal biopsies, and the like.

However, as mentioned above, the size of the additional device is typically small due to the progressively smaller sizes of endoscope 100, auxiliary scope 134 and the additional device. In examples, lumen 132 of endoscope 100 can typically be on the order of approximately 4.0 mm in diameter, while lumen 136 of auxiliary scope 134 can typically be on the order of approximately 1.2 mm. As such, with conventional devices, it can be difficult to obtain sufficiently large tissue sample sized to ensure accurate diagnoses without having to repeatedly remove and reinsert the additional device. Likewise, it can be difficult to view the desired matter, e.g., the target tissue, due to multiple reasons including the presence of the tissue retrieval device in the line of sight of the auxiliary scope camera. This thereby makes collection of non-desirable, e.g., non-cancerous, material a possibility. However, with the systems and devices of the present disclosure it is possible to obtain sufficiently large tissue sample sizes with only a single insertion and removal of the additional device, when configured as a tissue retrieval device or biopsy instrument of the present disclosure, for example. For example, the tissue retrieval device can be fabricated partially or entirely of translucent materials to allow imaging devices to have improved visibility of tissue behind the tissue retrieval device. Additionally, the tissue retrieval device can be fabricated partially or entirely of reflective materials to allow imaging devices to have improved visibility of particular components, e.g., functional components such as tissue cutters, of the tissue retrieval device. Furthermore, the present disclosure include tissue retrieval devices and biopsy devices that can be placed out front of the auxiliary scope and the lumen extending therethrough to increase the size and capacity of the tissue collection device.

FIG. 5A is a schematic illustration of surgical instrument 200 comprising elongate body 202, tissue collection device 204 and device controller 206. Surgical instrument 200 can comprise a device configured for the separation, collection and retrieval of biological matter, such as tissue, from a patient. Tissue collection device 204 can comprise separator 210, which, in the illustrated example, comprises jaws 212 and hinge 214 and activation mechanism 216. Controller 206 can comprise handpiece or handle 218, which can include activation mechanism 216 and connector 220. Elongate body 202 can comprise shaft 222 that can include lumen 224. Controller 206 can be connected to system control unit 16 (FIGS. 1 and 2) via cable 226 and the use of connector 220. The components illustrated in FIGS. 5A and 5B are not necessarily drawn to scale.

Tissue collection device 204 can be configured to do one or both of separate and retrieve biological matter from within a patient after being positioned within the patient by elongate body 202. Tissue collection device 204 can be configured to engage target tissue, separate the target tissue from the patient and store separated target tissue for removal from the patient, such as by removal of elongate body 202 from the patient.

Handpiece 218 can comprise any device suitable for facilitating manipulation and operation of surgical instrument 200. Handpiece 218 can be located at the proximal end of shaft 222 or another suitable location along shaft 222. In examples, handpiece 218 can comprise a pistol grip, a knob, a handlebar grip and the like. Actuation mechanism 216 can be attached to handpiece 218 to operate tissue collection device 204. Actuation mechanism 216 can comprise one or more of buttons, triggers, levers, knobs, dials and the like. Actuation mechanism 216 can be coupled to pressure-applying device 214 and can comprise any suitable device for allowing operation of pressure-applying device 214 from handpiece 218. As such, actuation mechanism 216 can comprise a linkage located within lumen 224 of shaft 222 or alongside shaft 222. In examples, the linkage can be a mechanical linkage, an electronic linkage or an electric linkage, (such as a wire or cable), or an activation energy source, such as an electric source, a fluid source or a gas source (such as a tube or conduit).

Shaft 222 can extend from handpiece 218 and can comprise an elongate member configured to allow tissue collection device 204 to be inserted into a patient. In examples, shaft 222 can be sized for placement within an auxiliary scope, such as scope 134 of FIG. 4. As such, shaft 222 can be inserted into an incision in the epidermis of a patient, through a body cavity of the patient and into an organ. Thus, it is desirable for the diameter or cross-sectional shape of shaft 222, as well as components attached thereto, to be as small as possible to facilitate minimally invasive surgical procedures. Tissue collection device 204 can thus be incorporated into shaft 222 to minimize the size impact on surgical instrument 200 and without interfering with the linkage. Shaft 222 can be axially rigid, but resiliently bendable, and formed from a metal or plastic material.

Tissue collection device 204 can be located at the distal end of shaft 222 or another suitable location along shaft 222. Tissue collection device 204 can be sized to fit within lumen 136 (FIG. 4), for example. Tissue collection device 204 can comprise a component or device for interacting with a patient, such as those configured to cut, slice, pull, saw, punch, twist or auger tissue, and the like. Specifically, separator 210 can comprise any device suitable for removing tissue from a patient, such as a blade, punch, scraping device or an auger. In additional examples, separator 212 can comprise a device configured to scrape or abrade tissue from the patient, such as a brush or grater device. In another example, separator 212 can comprise a roughened surface, such as a surface coated with hard particles, such as diamond or sand particles. Separator 210 can be configured to physically separate portions of tissue of a patient from other larger portions of tissue in the patient. In additional examples, separator 210 can be configured to simply collect biological matter from the patient that does not need physical separation, such as mucus or fluid. In examples, separator 212 can be configured to physically separate portion of tissue of a patient for retrieval with the tissue collection device or another device. In the illustrated example, separator 210 can comprise forceps having jaws 212 pivotably connected at hinge 214. Separator 210 can, however, be configured as a variety of devices capable of collecting biological matter, such as a punch, an auger, a blade, a saw and the like. Likewise, separator 210 can incorporate features for storing collected matter, such as a container or storage space. In an example, as discussed with reference to FIG. 6A, the storage space can be provided between jaws. Separator 210 can comprise forceps, as is described with reference to FIGS. 6A and 6B, or an auger, as is described with reference to FIGS. 7A and 7B. In any configuration, portions of separator 210 can be configured to allow light to pass therethrough or to reflect light incident thereon to selectively enhance images of separator 210 and anatomy obtained by an imaging unit.

Jaws 212 can be configured as a container or a walled element to hold and retain biological matter collected by tissue collection device 204. In an example, jaws 212 can comprise a flexible basket that can be deformed to allow portions of jaws 212 to be brought into close contact with target tissue. For example, jaws 212 can be fabricated from woven material such as strands of Kevlar, PVC, polyethylene, polycarbonate, PEEK and the like. Jaws 212 can be coupled to structural components, e.g., a frame, to facilitate coupling to shaft 222 and to facilitate mounting of cutting elements, such as teeth or blades, to jaws 212, as well as to provide stability for separator 210. In additional examples, jaws 212 can comprise a structural element, such as a box fabricated from rigid and inflexible material.

Handpiece 218 can be operated by a user to operate tissue collection device 204. Handpiece 218 can be used to manipulate shaft 222 to push separator 210 against target tissue. For example, shaft 222 can be rotated, oscillated, reciprocated and the like move separator 210 along the target tissue to cause separator 210 to separate sample tissue from the target tissue attached to the patient. Activation mechanism 216 can be coupled to handpiece 218 and can be configured to operate separator 210. Activation mechanism 216 can comprise any type of device suitable for activating the different types of separator devices described herein. In examples, activation mechanism 216 can comprise one or more of a lever, a trigger, a joystick, a button, a wheel and the like, as well as combinations thereof. In an example, activation mechanism 216 can comprise a wheel that can be rotated in one direction to open jaws 212 and rotated in an opposite direction to close jaws 212. For example, the wheel can be rotated to push and/or pull a wire to open and close jaws 212.

FIG. 5B is a close-up view of a distal end of tissue collection device 204 of FIG. 5A showing translucent tissue separator 210 extending from auxiliary endoscope 230. Endoscope 230 can comprise an example of auxiliary scope 134. Endoscope 230 can comprise shaft 232, working channel 234, passage 236 and lens 238. Field of view 240 can project from lens 238. Endoscope 230 can additionally include lens 239 for the projection of light into field of view 240.

Tissue collection device 204 can be configured as a low-profile device so as to be able to be inserted through a small diameter lumen, such as lumen 136 of auxiliary scope 134 of FIG. 4. Additionally, tissue collection device 204 can be configured as a high-capacity tissue collector that can hold a large volume of collected sample tissue to thereby reduce or eliminate the need to repeatedly remove surgical instrument 200 from the auxiliary scope. Furthermore, tissue collection device 204 can be optically enhanced to facilitate user operation of tissue collection device 204 to interact with target tissue. For example, jaws 212A and 212B can be fabricated from translucent material to allow lens 238 to see through jaws 212A and 212B, and teeth 213 can be fabricated of reflective material to reflect light from lens 239 back to lens 238 to allow a user to more clearly delineate where tissue collection device 204 will interact with target tissue of the patient. Jaws 212A and 212B can further be configured to provide magnification of target tissue when viewed through one or both of jaws 212A and 212B. In examples, one or both of jaws 212A and 212B can include one or more convex surfaces of transparent material to provide optical magnification.

Tissue collection device 204 can be fully retracted into working channel 234. Working channel 234 can comprise lumen 136 of FIG. 4. As such, lens 238 can be freely moved by manipulation of shaft 232 to position target tissue within field of view 240. However, when it is desired to extend tissue collection device 204 from working channel 234, tissue collection device 204 can become positioned within field of view 240, thereby inhibiting or preventing lens 238 from capturing images of the target tissue. As discussed with reference to FIGS. 6A-7B, tissue collection device 204 can be configured to allow light to 1) pass through components, portions or all of collector 210, and/or 2) be reflected by components, portions or all of collector 210 to enhance images obtained through lens 238.

FIG. 6A is a schematic illustration of surgical instrument 200 wherein separator device 210 comprises forceps 250 in a closed state and extended from endoscope 230 proximate target tissue 254. FIG. 6B is a schematic illustration of surgical instrument 200 with separator device 210 in a deployed state with forceps 250 open to engage target tissue 254. FIGS. 6A and 6B are discussed concurrently and the components therein are not necessarily drawn to scale.

As shown in FIG. 6A, tissue collection device 204 can be positioned in an anatomic duct 255 where target tissue 254 is located. Shaft 222 can be used to guide separator 210 through an anatomic duct to target tissue 254. Target tissue 254 can comprise a protrusion, such as a growth of cancerous or pre-cancerous material.

Endoscope 230 can be positioned such that lens 238 faces target tissue 254. As such, target tissue 254 can be within field of view 240 of lens 238. Field of view 240 is illustrated as having a particular viewing angle. However, lens 238 can be configured to have field of view 240 with different angles, up to and including one-hundred-eight degrees. As can be seen in FIG. 6A, tissue collection device 204 extended from shaft 232 to expose jaws 212A and 212B, but to not yet engage target tissue 254. As such, jaws 212A and 212B can thus be located to not completely block field of view 240 from target tissue 254. However, field of view 240 can become obstructed the further tissue collection device 204 becomes extended from working channel 234. For example, the portion of duct 255 from which target tissue 254 extends can become blocked from viewing by lens 238.

FIG. 6B is a side view of tissue collection device 204 with jaws 212 shown in cross-section to show storage space 256 with sample tissue 258. Jaws 212 can be elongated in the radial directions (e.g., up and down with respect to the orientations of FIG. 6B) so as to form a container for the storage of collected matter.

With jaws 212 rotated away from each other at hinge 214, tissue collection device 204 can be moved in the axial direction toward sample tissue 258. Jaws 212 can be rotated toward each other to engage target tissue 254. Tissue collection device 204 can be reciprocated back-and-forth along the axis of shaft 222 to collect sample tissue 258. Teeth 213 can be used to cut, saw, tear or rip portions of target tissue 254 away from the anatomy of the patient. In examples, only one of jaws 212A and 212B can be configured to rotate.

Teeth 213 can be fabricated out of an edge of jaws 212A and 212B. In examples, teeth 213 can comprise extensions of the material of jaws 212A and 212B. In such examples, both teeth 213 and jaws 212A and 212B can be fabricated of a rigid material such as plastic or metal. In examples, jaws 212A and 212B can be fabricated from Gorilla Glass @commercially available from Corning, or other chemically strengthened glass such as alkali-aluminosilicate sheet glass. In examples, jaws 212A and 212B can be fabricated from molded polycarbonate.

In additional examples, teeth 213 and jaws 212A and 212B can be mounted to a frame extending from hinge 214. For example, jaw 212A can comprise a U-shaped, rigid frame having end portions extending from hinge 214 to form a bounded space. Jaw 212A can comprise a bag or bellows of flexible material mounted to the U-shaped, rigid frame to partially enclose the bounded space. Teeth 213 can extend from the U-shaped, rigid frame away from the partially enclosed space. Jaw 212B can be configured similarly with teeth 213 configured to mesh with teeth 213 of jaw 212A. Thus, the flexible material of jaws 212A and 212B can form a full enclose when jaws 212A and 212B are rotate to engage, but can bend to not interfere with teeth 213 engaging target tissue 254.

Teeth 213 can be configured to have one or more orientations. For example, teeth 213 can be angled distally toward target tissue 254, or proximally toward shaft 222. In examples, some of teeth 213 can be angled proximally and some of teeth 213 can be angled distally. In examples, teeth 212 can be oriented in different directions.

As discussed above, components or portions of tissue collection device 204 can be made of optically enhanced materials. In examples, jaws 212A and 212B can be made of translucent or transparent material that can allow light waves to travel therethrough, thereby allowing lens 238 to “see through” jaws 212A and 212B. Transparent materials can allow lens 238 to see native coloring of target tissue 254. Translucent materials can be configured to allow lens 238 to see target tissue 254 in a filtered manner. As such, jaws 212A and 21B can be translucently tinted with different colors to enhance viewing of certain tissue types or mute viewing of other tissue types.

However, in order to maintain control of tissue collection device 204, e.g., to maintain accurate employment of teeth 213, portions of tissue collection device 204 can be opaque, reflective or translucent. In particular, teeth 213 can be made of opaque, reflective or translucent material or can have a coating applied thereto. In examples, teeth 213 can be opaque to be easily viewable by lens 238. In additional examples, teeth 213 can be configured to optically interact with light from lens 239. For example, teeth 213 can have a reflective coating applied thereto, such a coating of grains of reflective particles or titanium oxide. Thus, light from lens 239 can be bounced bac to lens 238. In additional examples, teeth 213 can be fluorescent to light up when engaged by a certain type of light. Thus, light from lens 239 can cause lens 238 to view teeth 213 in a particular wavelength that is more discernable relative to duct 255. In examples, only some of teeth 213 can be reflective or fluorescent.

In view of the foregoing, use of optically enhanced tissue collection devices can facilitate viewing of target tissue 254 through jaws 212A and 212B, viewing of sample tissue 258 within jaws 212A and 212B, and viewing of laceration 260 where sample tissue 258 was removed from target tissue 254. As such, endoscope 230 can be used to view interior tissue layers within laceration 260 and potentially diagnose conditions of the that tissue.

FIG. 7A is a schematic illustration of tissue retrieval device 300 comprising boring device 302, which can be inserted into endoscope 304. FIG. 7B is side view of tissue retrieval device 300 of FIG. 7A with boring device 302 shown in cross-section to show storage space 306 with sample tissue 308. FIGS. 7A and 7B are discussed concurrently and the components therein are not necessarily drawn to scale.

Tissue retrieval device 300 can further comprise shaft 310. Boring device 302 can comprise container 312, boring lands 314, blade 316 and bore 318. Endoscope 304 can be configured similarly as endoscope 230 of FIGS. 6A and 6B and can comprise another example of auxiliary scope 134. Endoscope 304 can comprise shaft 320, working channel 322, passage 324 and lens 326. Field of view 328 can project from lens 326. Endoscope 304 can further comprise light lens 329 for projecting light of one or more wavelengths onto target tissue 330.

Tissue retrieval device 300 can be configured to engage target tissue 330 in the axial direction of arrow B. For example, tissue retrieval device 300 can be positioned in front of a mound or protrusion of tissue or proximate a wall of tissue. Shaft 306 can be advanced in the direction of arrow B by a user to engage target tissue 330. Boring device 302 can be configured as a punch. Container 312 can have a cone shape and can include distal bore 318 that can be configured to push through tissue. Thus, tissue retrieval device can be configured to punch through tissue to take a tissue sample similar to core sampling a tree, etc. The distal or leading edge of bore 318 can be sharpened. In such a configuration, lands 314 and blade 316 can be omitted from container 312.

In examples, boring device 302 can be configured as an auger. As such, container 312 can have a cone shape with lands 314 wrapped around container 312 in a spiral manner. Lands 314 can be configured to engage tissue to allow container 312 to penetrate the tissue in the direction of arrow B. In some situations, it is possible for boring device 302 to slip over the target tissue, such as due to slippery or moist conditions. Thus, it can be difficult or impossible to engage the tissue sufficiently to collect a desirable volume of sample tissue. Lands 314 can be configured to facilitate engagement with the tissue. Shaft 306 can be rotated by an operator to rotate container 312 and lands 314. Lands 314 can grab tissue while being rotated to cause further axial penetration of boring device 302 into the tissue. As such, as boring device 302 is advanced forward, the distal tip of container 312 can maintain engagement with the tissue. As container 312 enters tissue, blade 316 can be configured to slice or shave tissue away from the patient. Blade 316 can comprise a sharpened edge of an opening in container 312 and can be configured similar to a potato peeler. In examples, only one of blade 316 and bore 318 can be used. However, both can be included as illustrated.

Additionally, In the various examples, container 312 can be configured to have an internal space to capture and retain sample tissue collected by bore 318 and/or blade 316.

As discussed herein, features of boring device 302 can be optically enhanced to interact with point of view of lens 326 and light being emitted at light lens 329. For example, container 312 can be fabricated from transparent or translucent material. As such, line of sight 340 can extend from lens 326 through container 312 to laceration 342 where sample tissue 308 was removed from target tissue 330. Additionally, line of sight 344 can extend from lens 326 through container 312 to sample tissue 308 within container 312.

Other features of boring device 302 can be configured to interact with light from lens 329. For example, boring lands 314, blade 316 and bore 318 can be fabricated from or coated with material to reflect light or to be luminescent.

Thus, as discussed herein boring device 302 can be optically enhanced to hide or make invisible portions of the device by being transparent or translucent and to visually brighten or highlight other portions of the device by being reflective or luminescent. Thus, portions of boring device 302, such as those not functionally important to identifying and removing target tissue, can be optically minimized to reduce noise in imaging signals for an operator, and portions of boring device 302, such as those that are functionally important to identifying and removing target tissue, can be optically maximized to increase visibility in imaging signals for an operator.

FIG. 8A is a schematic illustration of endoscopy system 400 comprising endoscope 402 and biopsy instrument 404. Biopsy instrument 404 can be tethered to the distal end portion of endoscope 402 for insertion into anatomy of a patient, thereby facilitating collection of large volumes of sample tissue without having to reinsert endoscope 402 into the patient multiple times.

Biopsy instrument 404 can comprise a device configured for the separation, collection and/or retrieval of biological matter, such as tissue, from a patient. In an example, biopsy instrument 404 can be configured as forceps shown in FIG. 8B. Biopsy instrument 404 can comprise separator 406, which, in the illustrated example, comprises jaws 408A and 408B, hinge 410, base 412, control cables 414A and 414B and couplers 416A and 416B. Biopsy instrument 404 can additionally comprise handpiece 418 and couplers 420A and 420B. Handpiece 418 can be operatively coupled to control unit 16 (FIGS. 1 and 2) via connector 421 and cable 419.

Endoscope 402 can comprise shaft 422, lumen 424, handpiece 426, control 428, connector 430 and cable 432. Handpiece 426 can comprise a controller for operating the functions of endoscope 402. For example, control 428 can comprise a knob for activating pull wires within shaft 422. Handpiece 426 can be connected to system control unit 16 (FIGS. 1 and 2) via cable 432 and the use of connector 430. The components illustrated in FIG. 8A are not necessarily drawn to scale.

Endoscope 402 can include components and features as are described with reference to endoscope 230 and endoscope 304 of FIGS. 5B-7B. Endoscope 402 can include steering capabilities (e.g., pull wires), illumination capabilities (e.g., a light emitter), guidance capabilities (e.g., a camera or imaging system) and fluid capabilities (e.g., irrigation and suction capabilities). In particular, endoscope 402 can be configured to operate with a working tool using lumen 424. Lumen 424 provides connection between distal-most end 434 of shaft 422 and handpiece 426 such that an instrument can be inserted into lumen 424 to function within anatomy through the distal end of shaft 422 and to be controlled at proximal end 436 of endoscope 402 via handpiece 426.

Biopsy instrument 404 can comprise a working tool configured to retrieve, remove and collect biological matter from within a patient. In the illustrated example, biopsy instrument 404 comprises forceps. However, other biopsy instruments or working tools can be used, such as boring device 302 of FIGS. 7A and 7B, as well as the other devices described herein.

Base 412 can comprise a component upon which to mount separator 406 and that can engage shaft 422. In examples, base 412 can be configured to abut distal-most end 434 to be held in place by control cables 414A and 414B. In other examples, base 412 can be configured to be coupled to distal-most end 434, such as via a threaded coupling, a protrusion that can be interference fit with lumen 424, a quick connect coupling or a magnetic coupling. Hinge 410 can comprise an axle or pivot point mounted to base 412 upon which one or both of jaws 408A and 408B can pivot. Jaws 408A and 408B can thus be mounted to hinge 410.

Control cables 414A and 414B can extend from jaws 408 and 408B through, alongside or around base 412 for extension into lumen 424. Control cables 414A and 414B can comprise various devices or components allowing for remote, e.g., proximal, control of biopsy instrument 404. In examples, control cables 414A and 414B can comprise wires or cables configured to pull on components of biopsy instrument 404. In the illustrated example, two control cables are shown for manipulation of jaws 408A and 408B. However, only one control cable can be used or more than two control cables can be used.

Proximal ends of control cables 414A and 414B can be provided with couplers 416A and 416, respectively. Couplers 416A and 416B can be connected with couplers 420A and 420B of handpiece 426. The union of couplers 416A and 416B with couplers 420A and 420B, respectively, can allow the transmission of actuation force through control cables 414A and 414B to biopsy instrument 404 from handpiece 426. Thus, handpiece 426 can be operated or can include button, knobs, levers and the like, to pull and push control cables 414A and 414B. In examples, couplers 416A and 416B can comprise plugs and couplers 420A and 420B can comprise sockets. In examples, couplers 416A and 416B can comprise loops or eyelets and couplers 420A and 420B can comprise latches, clips, hooks and the like, or vice versa.

Biopsy instrument 404 is shown in FIG. 8A as a mechanically actuated biopsy instrument. However, in examples, an electrically actuated biopsy instrument can be provided in which one or more control cables are configured to deliver at least one of power and control signals to the biopsy instrument. As such, the biopsy instrument can comprise an electrically activated device, e.g., via an electric motor or actuator. Correspondingly, handpiece 426 can comprise appropriate actuators for operating electrical components of such a biopsy device, such as buttons, switches and the like.

Typically, an endoscope is inserted into the anatomy of a patient and then the working tool is inserted through the endoscope. As such, as discussed above, the working tool, and particularly the distal, functioning end of the working tool, must be sized to fit within the lumen of the endoscope, which limits the size of the functional end and the working tool disposed thereat, as the working lumen is necessarily smaller than the cross-section of the endoscope. As mentioned above, a typical working tool lumen such as lumen 424 can be configured to have a diameter of approximately 1.2 mm.

With the devices and systems of the present disclosure, a working tool can comprise a functional element that is larger than a typical working tool lumen of an endoscope by providing a working tool that can be attached pre-insertion to the distal end of the endoscope. The working tool lumen can be used for the passage of control elements from the working tool that can be coupled proximally to a controller or handpiece for the working tool. The working tool can be sized larger than the working tool lumen and can extend radially, relative to the longitudinal axis of the endoscope, beyond the working tool lumen. To facilitate such capabilities, the working tool can include components that are fabricated of materials that allow for the passage of light (e.g., transparent or translucent materials) in order to minimize obstruction of imaging and illuminating capabilities of the endoscope.

Biopsy instrument 404 can be coupled to endoscope 402 via insertion of couplers 416A and 416B into lumen 424 at distal-most end 434. Couplers 416A and 416B can be extended through shaft 422 and handpiece 426 to extend from proximal end 436. Base 412 can be abutted to shaft 422 and, in examples, mounted thereto. Couplers 416A and 416B can be linked with couplers 420A and 420B of handpiece 418. Handpiece 418 can be mounted to handpiece 426 via any suitable coupling, such as threaded fasteners, snap fit couplers, hook and loop fastener material and the like. In an example, tension applied to control cables 414A and 414B between base 412 and handpiece 418 by the joining of couplers 416A and 416B and couplers 420A and 420B, can be sufficient to join biopsy instrument 404 and handpiece 418 to endoscope 402. Configured as such, separator 406 can be tethered to shaft 422. However, separator 406 can be attached with other tethering arrangements, such as those discussed herein with reference to base 412.

Once assembled, biopsy device 404 can be positioned at distal-most end 434 to be manipulated at a proximal end by a user. Jaws 408A and 408B can be sized larger than lumen 424, thereby having larger internal volumes that permit larger volumes of tissue samples to be acquired. In order to facilitate operation of biopsy device 406 that is larger than lumen 424, which can potentially obstruct lenses 238 and 239 (FIG. 6A), jaws 408A and 408B can be made of light transmitting material, as is described throughout the present application. Furthermore, in order to facilitate collection of a large volume of sample biological matter, e.g., via executing multiple collection operations (e.g., “bites”) with forceps comprising biopsy instrument 404.

FIG. 8B is a side view of forceps 438 suitable for use as a biopsy device of the present disclosure. Forceps 438 can comprise base 440, jaws 442A and 442B, hinge 444, actuators 446A and 446B and control wires 448A and 448B. Forceps 438 is described with reference to engagement with endoscope 230 of FIG. 5B for the sake of illustration of imaging lens 238 and illumination lens 239. Base 440 can be configured to engage shaft 232. Base 440 can abut the distal-most face of shaft 232 and can be configured to be taller than height H1 of working channel 234, thereby preventing base 440 being capable of entering working channel 234. In examples, base 440 can mate flush with shaft 232 to provide a stable connection to shaft 232, thereby inhibiting rocking or vibration, and allowing jaws 442A and 442B to firmly engage target tissue. As mentioned, base 440 can additionally be configured to be positively held in place relative to shaft 232 via a mechanical coupling or the like.

Hinge 444 can comprise a connection point for jaws 442A and 442B to couple to base 440. Hinge 444 can comprise a round pin or shaft over which corresponding bores in jaws 442A and 442B can be fit. Thus, jaws 442A and 442B can be configured to freely rotate on hinge 444. However, rotation of jaws 442A and 442B on hinge 444 can be controlled by control wires 448A and 448B. Control wires 448A and 448B can be coupled to actuators 446A and 446B, respectively, of jaws 442A and 442B. Actuators 446A and 446B can comprise levers extending at angle from jaws 442A and 442B relative to a centerline of working channel 234. Thus, control wires 448A and 448B can be operated by handpiece 418 to pull actuators 446A and 446B to rotate jaws 442A and 442B about hinge 444 to facilitate collection of tissue samples. In examples, control wires 448A and 448B can be pre-curved to impart rotational bias to actuators 446A and 446B to an open or closed position. However, in examples, actuators 446A and 446B can be provided with other biasing elements, such as springs. As such, pulling of control wires 448A and 448B can cause closing or opening of jaws 442A and 442B, as desired. As illustrated, jaws 442A and 442B can include teeth to facilitate cutting and tearing of tissue away from the anatomy. Though the illustrated example is shown with reference to actuators comprising levers, other actuators, such as pull rods or screw mechanisms, can be used.

As illustrated, jaws 442A and 442B can extend radially beyond height H1 of working channel 234 so as to obstruct lenses 238 and 239. In an example, working channel 234 can have height H1 of 1.2 mm. In particular, jaw 442A can extend radially above working channel 234 to be positioned between lenses 238 and 239 and target tissue distal of endoscope 230. As such, in order to prevent jaws 442A and 442B from preventing lenses 238 and 239 from providing guidance and target tissue acquisition to endoscope 230, such as by providing imaging of tissue, jaws 442A and 442B can be made of material that allows light to pass therethrough, such as transparent, translucent and semi-opaque material, as is described herein. As such, jaws 442A and 442B can be larger than working channel 234 without interfering with operation of endoscope 230.

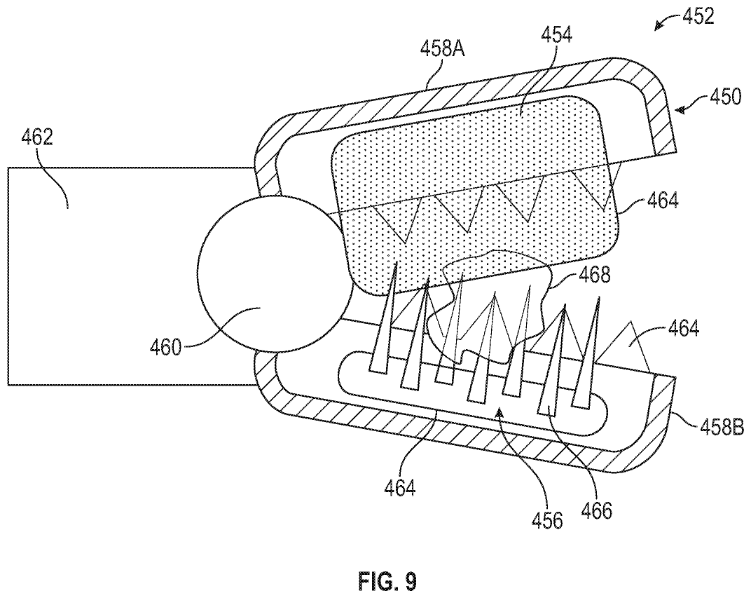

FIGS. 9-11 illustrate examples of a biopsy instrument suitable for use with the present disclosure. FIGS. 9-11 illustrate simplified schematic views of forceps 438 of FIG. 8B. However, other tissue collection or retrieval devices and other forceps configurations can be used.

FIG. 9 is a schematic illustration of biopsy instrument 450 comprising forceps 452 having a tissue retention system comprising sponge 454 and needle array 456. Forceps 452 can comprise jaws 458A and 458B, hinge 460 and base 462. Jaws 458A and 458B can include teeth 464. Needle array 456 can comprise base 464 and needles 466. Tissue sample 468 can be located between jaws 458A and 458B. Sponge 454 can comprise a resiliently deformable body that can be deformed by the presence of sample tissue within jaws 458A and 458B, but that tends to retain its shape to apply a retaining force to the sample tissue. Needles 466 can comprise tines or pins configured to pierce sample tissue and sponge 454.

Sponge 454 and needle array 456 can comprise a capacity enhancement feature that allows jaws 458A and 458B to hold a larger volume of sample tissue than without sponge 454 and needle array 456. Sponge 454 can be attached to the internal cavity of jaw 458A, such as via adhesive or any suitable manner, and used to bias tissue sample 468 toward 458B. Base 464 can be attached to the internal cavity of jaw 458B, such as via adhesive or any suitable manner. As such, jaws 458A and 458B can be used to obtain tissue sample 468 and position tissue sample 469 between jaws 458A and 458B, such as by using control wires 448A and 448B. Thereafter, jaws 458A and 458B can be reopened to obtain an additional tissue sample, and sponge 454 can push tissue sample 468 into needles 466 to prevent tissue sample 468 from falling out of forceps 452. In examples, sponge 454 and needle array 456 can be used independently (e.g., one without the other) to retain tissue sample 468 between jaws 458A and 458B.

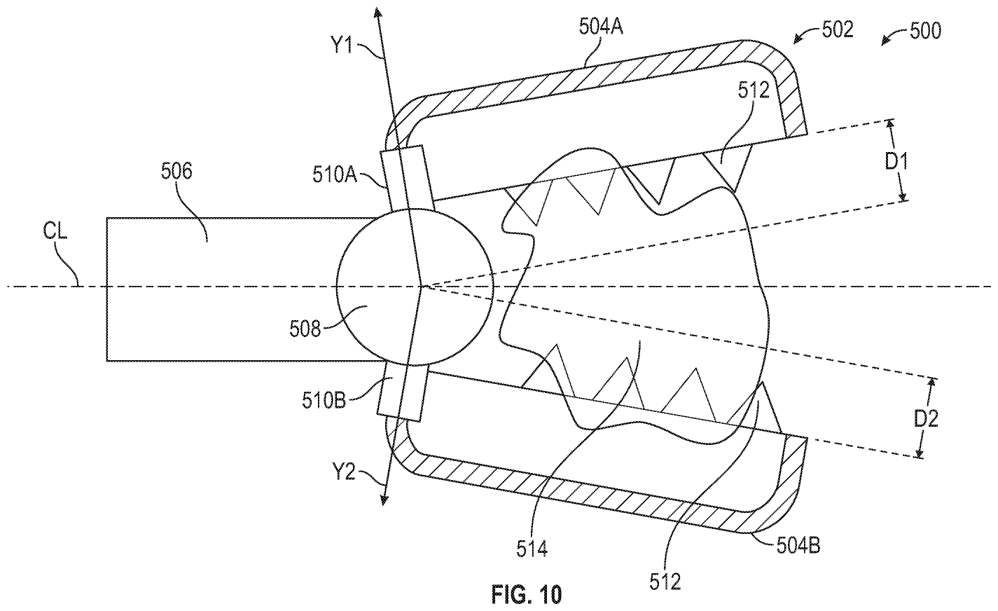

FIG. 10 is schematic illustration of biopsy instrument 500 comprising forceps 502 having expandable jaws 504A and 504B. Forceps 502 can comprise base 506, hinge 508 and rails 510A and 510B. Jaws 504A and 504B can include teeth 512. Tissue sample 514 can be located between jaws 458A and 458B. Jaw 504A can be slidably coupled to rail 510A so as to be displaceable in direction Y1. Thus, jaw 504A can be displaced distance D1 from centerline CL (relative to not being rotated). Similarly, Jaw 504B can be slidably coupled to rail 510B so as to be displaceable in direction Y2. Thus, jaw 504B can be displaced distance D2 from centerline CL (relative to not being rotated).

Jaws 504A and 504B can be used to obtain tissue sample 514, such as via actuation by control wires 448A and 448B. Jaws 504A and 504B can be moved radially outward in the direction of arrows Y1 and Y2. In an example, jaws 504A and 504B can be moved on rails 510A and 510B by resistance from tissue sample 514. Jaws 504A and 504B can include tracks that ride in rails 510A and 510B. Thus, upon the presence of tissue sample 514 when jaws 504A and 504B are being actuated to be closed, jaws 504A and 504B can move outwardly to accommodate the presence of tissue sample 514. The tracks can ride in rails 510A and 510B with an appropriate level of friction to prevent free movement therebetween. Jaws 504A and 504B can thus be moved to accommodate the collection of multiple tissue samples or larger sized samples as compared to jaws that are fixed at the pivot point.

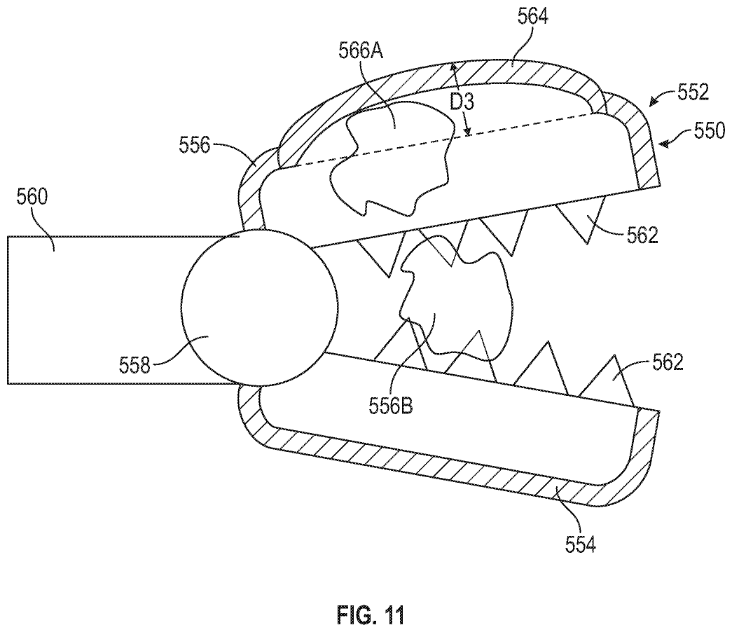

FIG. 11 is a schematic illustration of biopsy instrument 550 comprising forceps 552 having flexible jaw 554 and opposing jaw 556. Flexible jaw 554 and opposing jaw 556 can be connected at hinge 558 and coupled to base 560. Jaws 554 and 556 can include teeth 562. Flexible jaw 554 can comprise deflectable wall 564. Tissue samples 566A and 566B can be located between jaws 554 and 556.

Jaws 554 and 556 can be used to obtain tissue sample 556A, such as via actuation by control wires 448A and 448B. Thus, tissue sample 556A can be positioned between jaws 554 and 556. Tissue sample 556A can occupy the space between jaws 554A and 554B. However, rather than stopping the tissue collection procedure to withdraw biopsy instrument 550 and the endoscope in which it is inserted, jaws 554 and 556 can be operated to collect second tissue sample 556B, which can be positioned between jaws 554A and 556. The presence of tissue sample 556B can displace tissue sample 556A outward toward jaw 556. Tissue sample 566A can deflect deflectable wall 564 outward away from hinge 558, distance D3 from an undeflected position, to produce more space between jaws 554 and 556.

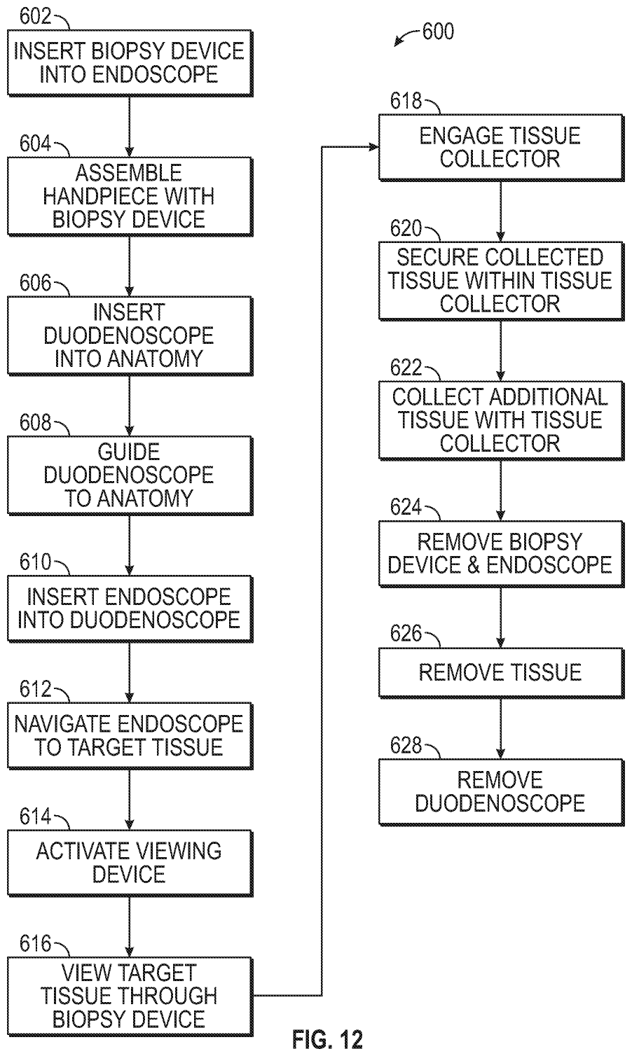

FIG. 12 is a block diagram illustrating examples of method 600 of collecting biological matter from a patient using the biopsy devices and tissue retrieval devices of the present disclosure, such as those that are tethered distally of an endoscope. Method 600 can encompass the use of endoscopy system 400 of FIG. 8A, forceps 438 of FIG. 8B, biopsy instrument 450 of FIG. 9, biopsy instrument 500 of FIG. 10 and biopsy instrument 550 of FIG. 11, as well as other instruments including those described herein.

At step 602, a biopsy device, such as forceps 438 of FIG. 8B, biopsy instrument 450 of FIG. 9, biopsy instrument 500 of FIG. 10 and biopsy instrument 550 of FIG. 11, can be inserted into endoscope 402. For example, control cables 414A and 414B can be inserted into lumen 424 of shaft 422. In examples, control cables 414A and 414B can be longer than endoscope 402 such that proximal ends of control cables 414A and 414B having couplers 416A and 416B can extend proximally out of endoscope 402.

At step 604, the biopsy device can be attached to the endoscope to prevent separation therefrom. For example, handpiece 418 can be assembled to handpiece 426 to prevent control cables 414A and 414B from sliding out of lumen 424, such as by attaching couplers 420A and 420B to couplers 416A and 416B. In other examples, couplers 416A and 416B can be attached to handpiece 426 without the use of handpiece 418. Additionally, base 412 of biopsy device 406 can be attached to shaft 422 of endoscope 402.

At step 606, the duodenoscope can be inserted into anatomy of a patient, such as by being inserted into an opening or incision in the patient. In examples, the duodenoscope can be guided to a duodenum of the patient to perform a cholangioscopic procedure. However, the tethered biopsy devices of the present disclosure can be used in other types of procedures referenced herein, such as other gastrointestinal procedures and renal area procedures.

At step 608, the duodenoscope can be inserted into and navigated through anatomy of a patient. For example, endoscope 14 (FIG. 1) can utilize native imaging capabilities to guide insertion section 28 through anatomic ducts of the patient. Insertion section 28 can be bent or curved using control knob 38 to facilitate turning of endoscope 14.

At step 610, an endoscope or auxiliary scope can be inserted into the duodenoscope to access anatomy located further in the duct. For example, endoscope scope 402 (FIG. 8A), with biopsy device 404 attached thereto, can be inserted into lumen 62 (FIG. 3C) or lumen 132 (FIG. 4) to reach another anatomic duct intersecting the anatomic duct reached by endoscope 14. Elevator 54 (FIG. 3C) can be used to bend or turn endoscope 402.

At step 612, the endoscope can be navigated through the anatomy. For example, endoscope 402 can be guided from the duodenum to the common bile duct. The endoscope can be guided using native steering and imaging capabilities of the endoscope.

At step 614, a viewing device or imaging device on the auxiliary scope can be activated in order to view biological matter of the patient. For example, imaging unit 110 can be activated to view anatomy in field of view 240 of lens 238. Images can be sent back to control unit 16.

At step 616, target tissue can be viewed using an imaging unit and a video display monitor. For example, imaging unit 110 can use lens 238 to display target tissue on output unit 18. Lens 238 can view the target tissue through transparent or translucent portions of the tissue collection devices, such as forceps 438 of FIG. 8B, biopsy instrument 450 of FIG. 9, biopsy instrument 500 of FIG. 10 and biopsy instrument 550 of FIG. 11. Light from a light source can be used to illuminate the target tissue. For example, light from lens 239, as generated by lighting unit 112, can be directed upon the target tissue. As discussed herein, various components of the tissue collection devices can be configured to reflect light from the light source to enhance visibility.

At step 618, a tissue collector of the biopsy device can be navigated to the location of target tissue within the patient. For example, jaws 408A and 408B can be navigated through an anatomic duct to target tissue 254 (FIG. 6A). The target tissue can comprise tissue that is potentially diseased or otherwise indicative of a diseased condition of the patient. Jaws 408A and 408B can be pushed, pressed or otherwise brought into pressurized contact with the target tissue. Thus, jaws 408A and 408B can be rotated about hinge 410 by activation of guide cables 414A and 414B from handpiece 418 to cause jaws 408A and 408B to slice, punch, shave, etc. one or more pieces of tissue away from the anatomy of the patient. Furthermore, portions of the tissue collection device can interact with light from lens 239 to enhance visibility of such portions. For example, tissue separating components, such as teeth or blade edges, can be reflective or luminescent to enhance display on the video display monitor, such as output unit 18.

At step 620, sample tissue or biological matter separated or collected from the patient at step 618 can be stored within a space or internal volume inside the tissue collection device. For example, separated sample tissue 258 can be positioned within space 256. As explained with reference to biopsy instrument 450 of FIG. 9, biopsy instrument 500 of FIG. 10 and biopsy instrument 550 of FIG. 11, the tissue retrieval devices can include capacity enhancing features that facilitate the collection of large volumes of sample tissue, such as multiple sample tissue volumes. Sponge 454 and needle array 456 can comprise retention features that can operate independently or cooperatively to bias or retain collected tissue samples within jaws 458A and 458B to prevent dislodging of the collected tissue samples. Slidable rails 510A and 510B, and deflectable wall 564 can comprise capacity increasing features that can be employed to secure increasingly larger volumes of tissue within their respective jaws.

At step 622, additional tissue can be collected with the biopsy device by reapplying the tissue separator device. As more tissue pieces are collected, the newly collected pieces can push the previously collected pieces further into the tissue retrieval device. The previously collected pieces can then activate the capacity enhancing features, such as by the previously collected pieces being pushed into engagement with sponge 454, being pushed into needle array 456, pushing movable jaws 504A and 504B outward, and moving deflectable wall 564.

At step 624, the biopsy device can be removed from the patient, such as by removal from the duodenoscope, which can be left in place inside the anatomy. Safeguards can be put into place to ensure removal of the tissue collection device without inadvertently cutting anatomy of the patient.

At step 626, the collected sample tissue can be removed from the tissue collection device. For example, jaws 408A and 408B can be rotated away from each other to access space therebetween and remove sample tissue for analysis, etc.

At step 628, the duodenoscope can be removed from the patient. The patient can thereafter be appropriately closed up or prepared for completion of the procedure.

As such, method 600 illustrates examples of a method of collecting biological matter from internal passages of a patient in large enough quantities, e.g., by using an optically enhanced (e.g., transparent, clear, reflective, translucent, luminescent, or scattering) tethered tissue removal device with internal storage, to eliminate or reduce insertion and removal of surgical devices from the patient. Tethering of the tissue removal device allows for a larger instrument to be used than the working channel or lumen of an endoscope can allow. The optical enhancements allow the tissue removal device to be at least partially invisible to a camera and recognized by a light source, for example.

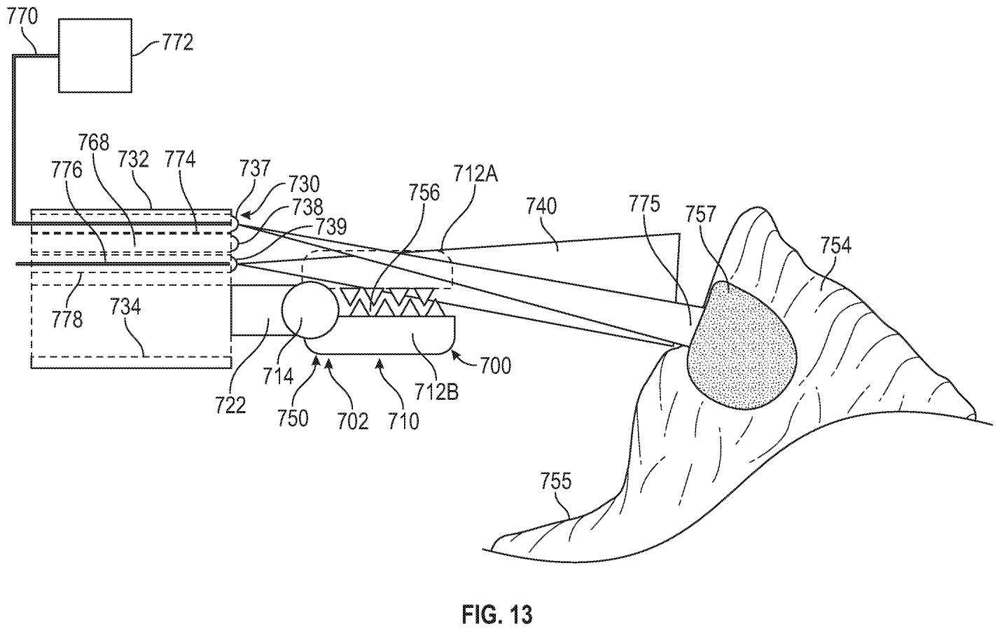

FIG. 13 is a schematic illustration of a surgical instrument 700 comprising collection device 702 and laser fluorescence capabilities, such as lens 737. Collection device 702 can comprise tissue separator 710 comprising forceps 750 including jaws 712A and 712B connected at hinge 714. FIG. 13 shows forceps 750 extended from working channel 734 of shaft 732 of endoscope 730 proximate target tissue 754. Components of FIG. 13 are not necessarily drawn to scale. FIG. 13 is described with reference to endoscope 730 and collection device 702, but the laser fluorescence and other light-energizing capabilities described herein can be utilized with other scopes and surgical instruments, including colonoscopes, hysteroscopes, uretoscopes, laparoscopes and duodenoscopes. Surgical instrument 700 can comprise a standalone instrument that can be used by itself, with or without a mother scope, such as a duodenoscope.

Collection device 702 can be positioned in an anatomic duct 755 where target tissue 754 is located. Shaft 722 can be used to guide separator 710 through anatomic duct 755 to target tissue 754. Target tissue 754 can comprise a protrusion, such as a growth of cancerous or pre-cancerous material. Target tissue 754 can include dye material 757. Dye material 757 can comprise fluorescent dye or another material capable of emitting light after absorbing an energization light.

Imaging lens 738 can comprise an imaging component that can be connected other optical components, such as a prism and an imaging unit (e.g., imaging unit 67 of FIG. 3B), which can be coupled to wiring 768 that can extend, directly or indirectly (via handle section 32 and/or other wiring), to control unit 16 (FIG. 2). Wiring 768 can be similar to wiring 68 of FIG. 3B.

Energizing lens 737 can comprise a first light emitter from which a first light can be emitted. Lens 737 can be connected to light transmitter 770, which can comprise a fiber optic cable or cable bundle or another light transmitter extending to light source 772 through passage 774. Light source 772 can be connected to control unit 16 (FIG. 2) via handle section 32 and/or other wiring or connections. Thus, light from light source 772 can be transmitted through light transmitter 770 to lens 737 such that energization light 775 is produced.

Illumination lens 739 can comprise a second light emitter from which a second light can be emitted. Lens 739 be connected to light transmitter 776, which can comprise a fiber optic cable or cable bundle extending to light source 22 (FIG. 1) that can extend, directly or indirectly (via handle section 32 and/or other wiring or connections), to control unit 16 (FIG. 2). Light transmitter 764 can extend through passage 778. Thus, light from light source 22 can be transmitted through light transmitter 776 to lens 739 such that illumination light 740 is produced.

As discussed below, the combination of energization light from lens 737 and illumination light from lens 739, as well as light transmitting properties (e.g., transparency) of jaws 712A and 712B, can allow for better viewing of target tissue 754 with the aid of light-energized dye material 757. Light source 772 can be configured to emit light to illuminate tissue and energize dye within the tissue so as to facilitate better acquisition of target tissue 754 with jaws 712A and 712B and better viewing by lens 738.

Endoscope 730 can be positioned such that lens 738 faces target tissue 754. As such, target tissue 754 can be within the field of view of illumination light 740 of lens 739. Field of view of illumination light 740 is illustrated as having a particular viewing angle. However, lens 739 can be configured to have a field of view with different angles, up to and including one-hundred-eighty degrees.

Target tissue 754 can be within the field of view of energization light 775 of lens 737. The field of view of energization light 775 can be up to one-hundred-eighty degrees. However, the field of view of energization light 775 can be more focused to only illuminate a smaller area of duct 755. For example, the field of view of light 775 can be smaller than the field of view of light 740. The field of view of light 775 can be configured to project to about the size of tissue collection device 702 at a length tissue collection device 702 is desired to be operated. As such, tissue energized by energization light 775 can provide a direction finder for tissue separator 710 to extend.

Tissue collection device 702 can be extended from shaft 732 to expose jaws 712A and 712B and reach target tissue 754. As such, jaws 712A and 712B can be located within illumination light 740 and energization light 775. Jaws 712A and 712B can therefore block or obstruct light 740 and light 775 from reaching target tissue 754. For example, the portion of duct 755 from which target tissue 754 extends can become blocked from viewing by lens 738. As such, as described herein, jaws 712A and 712B can be fabricated of material that can at least partially let light waves pass therethrough, including clear, transparent and translucent materials. The material of jaws 712A and 712B can additionally include material or substances that can be energized by energization light 775 or reflected by illumination light 740 to enhance viewing by imaging lens 738. In examples, tissue collection device 702 can be fabricated of opaque materials or non-light transmitting materials. For example, tissue collection device can be sized to fit through working channels or lumens of scopes without, or with reduced potential for, obstructing viewing of imaging devices whereby the desirability of transparency of lower.

Light that passes through jaws 712A and 712B can be incident on target tissue 754. Illumination light 740 can provide visible light to aid in viewing of tissue. Energization light 775 can provide other light to energize dye material 757. Dye material 757 can be energized by light from lens 737 to facilitate identification of and retrieval of target tissue 754.

Dye material 757 can comprise one or more surgical dyes including fluoroscopic and near-infrared dyes. In examples, dye material 757 can comprise a luminescent material. In examples, dye material 757 can comprise or include a fluorophore, which is a fluorescent chemical compound that can re-emit light upon light excitation. In examples, dye material 757 can comprise blue dyes (methylene blue) that can be used in cancer surgeries and fluorescent dyes, such as indocyanine green (ICG), that can be used in endometriosis surgeries. In an example, dye material 757 can comprise fluorescein (maximum excitation at 490 nm) and light source 772 can comprise an Argon-Ion, Blue-Green laser which predominantly emits wavelengths 488 (blue) and 514 (green) nm.

TABLE 1

| | | Peak Wavelength |
|---|---|---|
| Dye | Treatment | (nm) Absorption |
| SGM-101 | Colon Cancer | 685 |
| EMI-137 | Colon Cancer | 650 |
| LUM-015 | Breast Cancer | 650 |
| LUM-015 | Colon, Pancreatic, Esophageal | 650 |
| | Cancer | |
| ICG | Tissue Perfusion | 805 |
| OTL-38 | Ovarian Cancer | 775 |
| BLZ-100 | Pediatric Central Nervous | 805 |
| | System Tumors | |
| OTL-38 | Lung Cancer | 775 |
| AVB-620 | Breast Cancer | 750 |
| IRDye-800BK | Ureter | 775 |
| Cet.-IRDye800 | Head and Neck | 775 |
| Beva.-IRDye800 | Pancreatic | 775 |
| Beve.-IRDye800 | Endometriosis, Rectal Cancer | 775 |
| ABY-029 | Head and Neck | 770 |
| VM-110 | Ovarian, Pancreatic Cancer | 750 |

Table 1 shows various dyes, associated tissues where such dyes can be used and the wavelength for peak absorption of such dyes. As such, lasers and other light sources that can emit light having wavelengths around the peak absorption can be used to energize the listed dyes.

In examples, light 775 can comprise infrared light, near-infrared light or ultraviolet light. In examples, light 775 can blue or green light. In examples, light 775 can comprise light having a wavelength in the range of approximately 400 to 800 nanometers (nm). Light 775 from light source 772 can provide excitation energy absorbed by molecules of the tissue to activate a luminescent dye administered to the patient. Light source 772 can be configured as a laser or can produce light amplification by stimulated emission of radiation. In examples, laser light can be produced in the range of 358-405 nanometers.

Light source 772 can comprise a stand-alone module couplable to surgical instrument 700 via light transmitter 770. As such, light source 772 can be located remotely from surgical instrument 700. In additional examples, light source 772 can be attached directly to the exterior of handle section 32 (FIG. 2). Light source 772 can be removably attached to handle section 32 via a coupler, thereby allowing for attachment of light generators that produce different intensities or wavelengths which can allow for energization of different types of surgical dyes including fluoroscopic and near-infrared dyes. In additional examples, light source 772 can be incorporated into handle section 32 such that a connector is not used. In additional examples, light source 772 can be incorporated into control unit 16 and light transmitter 770 and cable section 34 (FIG. 1) can be included in a common cable bundle.

Light transmitter 770 can connect light source 772 with the distal end portion of shaft 732, such as at lens 737. Light transmitter 770 can comprise one or more cables or conductors capable of conducting, communicating or transmitting light waves. In examples, light transmitter 770 can comprise fiber optic cables. In examples, the fiber optic cables can comprise glass and plastic fibers jacketed with one or more protective and reflective coatings. Light transmitter 770 can be disposed within passage 774. In examples, light transmitter 770 can be embedded in shaft 732 or can be located in a channel provided therein. In examples, light transmitter 770 can be located on the exterior of shaft 732 and secured thereto via a sheath. In examples, light transmitter 770 can be glued or adhered to shaft 732, either on the interior or exterior.

Lens 737 can be located at or near the distal end of transmitter 770. Lens 737 can be coupled to light transmitter 770 by any suitable means. In examples, lens 737 can comprise a lens for collecting and focusing light waves from light transmitter 770. Lens 737 can comprise a glass or plastic body of transparent material. However, in additional examples, a separate light emitter is not used and light transmitter 770 can comprise an end-emitting fiber such that the distal or terminal end of light transmitter 770 can comprise a light emitter.

In examples, light 740 can comprise incandescent light from a light bulb or directional light from a light-emitting-diode. In examples, light 740 can white light or yellow light. In examples, light 740 can comprise light having a wavelength in the range of approximately 400 to 700 nanometers (nm). Light 740 can be visible to the naked eye to facilitate imaging lens 738 obtaining images of duct 755 that can be viewed at output unit 18 (FIG. 1).

With light 740 illuminating duct 755 and light 775 energizing dye material 757, tissue separator 710 can be engaged with target tissue 754. Jaws 712A and 712B can be rotated away from each other at hinge 714, and tissue collection device 702 can be moved in the axial direction toward sample tissue 758. Jaws 712A and 712B can be rotated toward each other to engage target tissue 754. Tissue collection device 702 can be reciprocated back-and-forth along the axis of shaft 722 to collect sample tissue 758. Teeth 713 can be used to cut, saw, tear or rip portions of target tissue 754 away from the anatomy of the patient. In examples, only one of jaws 712A and 712B can be configured to rotate. As mentioned, energized dye material 757 can provide a target to which jaws 712A and 712B can be navigated.

Teeth 713 can be fabricated out of an edge of jaws 712A and 712B. In examples, teeth 713 can comprise extensions of the material of jaws 712A and 712B. In such examples, both teeth 713 and jaws 712A and 712B can be fabricated of a rigid material such as plastic or metal. In examples, jaws 712A and 712B can be fabricated from Gorilla Glass @commercially available from Corning, or other chemically strengthened glass such as alkali-aluminosilicate sheet glass. In examples, jaws 712A and 712B can be fabricated from molded polycarbonate.

As discussed above, components or portions of tissue collection device 702 can be made of optically enhanced materials. In examples, jaws 712A and 712B can be made of translucent or transparent material that can allow light waves to travel therethrough, thereby allowing lens 738 to “see through” jaws 712A and 712B. Transparent materials can allow lens 738 to see native coloring of target tissue 754. Translucent materials can be configured to allow lens 738 to see target tissue 754 in a filtered manner. As such, jaws 712A and 712B can be translucently tinted with different colors to enhance viewing of certain tissue types or mute viewing of other tissue types.

However, in order to maintain control of tissue collection device 702, e.g., to maintain accurate employment of teeth 713, portions of tissue collection device 702 can be opaque, reflective or translucent. In particular, teeth 713 can be made of opaque, reflective or translucent material or can have a coating applied thereto having those properties. In examples, teeth 713 can be opaque to be easily viewable by lens 738. In additional examples, teeth 713 can be configured to optically interact with light from lens 739. For example, teeth 713 can have a reflective coating applied thereto, such a coating of grains of reflective particles or titanium oxide. Thus, light from lens 739 can be bounced back to lens 738. In additional examples, teeth 713 can be fluorescent to light up when engaged by a certain type of light, such as light from lens 737. Thus, light from lens 739 can cause lens 738 to view teeth 713 in a particular wavelength that is more discernable relative to duct 755. In examples, only some of teeth 713 can be reflective or fluorescent.

In view of the foregoing, use of optically enhanced tissue collection devices can facilitate viewing of target tissue 754 through jaws 712A and 712B, viewing of sample tissue 758 within jaws 712A and 712B, and viewing of a laceration where sample tissue 758 was removed from target tissue 754. As such, endoscope 730 can be used to view interior tissue layers within the laceration and potentially diagnose conditions of the that tissue.

In additional examples, teeth 713 and jaws 712A and 712B can be configured similarly as teeth 213 and jaws 212A and 212B as described with reference to FIGS. 6A and 6B, as well as the devices shown and described with reference to FIGS. 9, 10 and 11. For example, teeth 713 can be oriented in forward or back ward orientations relative to the interior of jaws 712A and 712B, and jaws 712A and 712B can include features to facilitate increased capacity or holding capability of collected tissue samples, such as by being expandable.

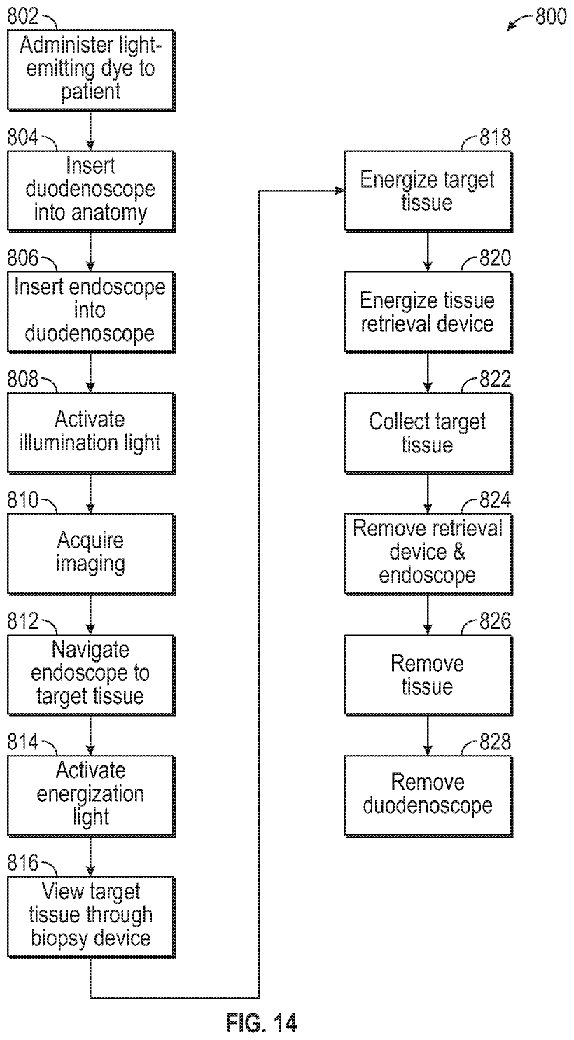

FIG. 14 is a block diagram illustrating examples of method 800 of collecting biological matter from a patient using the tissue collection and tissue retrieval devices of the present disclosure, such as those that include light-emitting capabilities to activate light-emitting materials, as described herein. Method 800 can encompass the use of endoscopy system 700 of FIG. 13, as well as other instruments including those described herein. In examples, surgical instruments such as cutting forceps having cauterizing and ablating capabilities in which the light-emitting capabilities and light-emitting materials of the present disclosure can be utilized are described in U.S. patent application Ser. No. 17/100,025 to Murdeshwar, titled “Surgical instruments with integrated lighting systems” which is hereby incorporated by this reference in its entirety. Method 800 of FIG. 14 is described with reference to FIG. 13, unless otherwise specifically noted.

At step 802, a dye can be administered to a patient. The dye can be ingested or administered intravenously. The dye can comprise any type of dye or dye material used for surgical procedures as discussed herein. The dye or other material can be configured to absorb light of a first wavelength from a light source and emit light of a second wavelength. As discussed herein, the dye can comprise fluoroscopic material, luminescent material and the like. The dye can be metabolized or otherwise injected or absorbed into the tissue of the patient, including by tissue that is to be targeted by the surgeon for abatement, e.g., target tissue 754. The tissue of a uterus, a bladder, ovaries and other locations and organs, such as fallopian tubes and the rectum, can metabolize the dye. The target tissue can be cancerous tissue or endometriotic tissue. Step 800 can alternatively be performed pre-operatively or intraoperatively.

At step 804, a duodenoscope (e.g., scope 14 of FIG. 1) can be inserted into anatomy of a patient, such as by being inserted into an opening or incision in the patient. In examples, the duodenoscope can be guided to a duodenum of the patient to perform a cholangioscopic procedure. However, the present disclosure can be utilized with other devices and procedures used in other types of procedures referenced herein, such as other gastrointestinal procedures, renal area procedures and cancer treatment procedures. The duodenoscope can be inserted into and navigated through anatomy of a patient. For example, endoscope 14 (FIG. 1) can utilize native imaging capabilities to guide insertion section 28 through anatomic ducts of the patient. Insertion section 28 can be bent or curved using control knob 38 to facilitate turning of endoscope 14.

At step 806, another scope can be inserted into the duodenoscope. An endoscope or auxiliary scope can be inserted into the duodenoscope to access anatomy located further in the duct. For example, endoscope 730 along with tissue collection device 702 (FIG. 13) can be inserted into lumen 62 (FIG. 3C) or lumen 132 (FIG. 4) to reach another anatomic duct intersecting the anatomic duct reached by endoscope 14. Elevator 54 (FIG. 3C) can be used to bend or turn endoscope 402.

At step 808, illuminating light 740 can be emitted. For example, a light source capable of emitting visible light can be used to provide illumination of anatomy distal of the auxiliary scope. Lighting unit 112 (FIG. 4) can be activated by a switch on handle section 32 (FIG. 2) to illuminate anatomy distal of forceps 750. Thus, visible light can be emitted to aid viewing of imaging unit 110 (FIG. 4). For example, light from lens 739, as generated by lighting unit 112, can be directed upon the target tissue.

At step 810, a viewing device or imaging device on the auxiliary scope can be activated in order to view biological matter of the patient. For example, imaging unit 110 can be activated to view anatomy in field of view of illumination light 740 of lens 738. Images can be sent back to control unit 16 (FIG. 2). Target tissue 754 and surrounding tissue can be viewed using an imaging unit and a video display monitor. For example, imaging unit 110 can use lens 738 to display target tissue on output unit 18 (FIG. 1).

At step 812, endoscope 730 can be navigated through the anatomy. For example, endoscope 730 can be guided from the duodenum to the common bile duct. Endoscope 730 can be guided using native steering and imaging capabilities of the endoscope. Once the auxiliary scope is in the general region of the anatomy where the target tissue is located, activation light 775 can be emitted to reach the specific location of the target tissue.

At step 814, energizing light 775 can be emitted. Light source 772 can be capable of emitting light at a wavelength compatible with the dye administered at step 802. Light 775 can be used to energize dye material 757. Light source 772 can be activated by a switch on handle section 32 (FIG. 2) to interact with anatomy distal of forceps 750. Thus, energizing light 775 can be emitted to aid identification of target tissue 754. For example, light from lens 737, as generated by light source 772, can be directed upon target tissue 754.

At step 816, target tissue 754 can be viewed through tissue collection device 702. Lens 738 can view the target tissue through transparent or translucent portions of tissue collection device 702, such as jaws 712A and 712B of forceps 750 of FIG. 13. At any point during the procedure, visible wavelength light from lens 738 can pass through collection device 702. Light 740 and light 775 can be toggled on and off as needed by a user to use neither, both or just one of light 740 and light 775.

At step 818, portions of the anatomy in which the dye of step 802 has been metabolized can interact with light from lens 737. The light can additionally be light of a wavelength sufficient to energize, e.g., fluoresce, the dye, such as near-infrared (NIR) light. NIR light can be used to energize indocyanine green in endometriosis surgeries. NIR light is typically located in the near-infrared region of the electromagnetic spectrum, from approximately 780 nm to approximately 2500 nm. The indocyanine green dye can concentrate in vascular rich areas, such as in endometrium tissue. Additionally, the combinations of light and dye listed in Table 1 can be utilized together. Light 775 can energize dye metabolized into tissue of duct 775. In particular, damaged, diseased or otherwise undesirable tissue can metabolize the dye in such a way that light 775 will allow that tissue to be more readily distinguished from neighboring tissue. Energized dye can be viewed by camera lens 738 connected to imaging unit 110 (FIG. 4). Video from lens 738 can be viewed by an eyepiece attached to endoscope 730 or at output unit 18 (FIG. 1), which can comprise a video monitor. For example, target tissue 754 can include dye material 757, indicating target tissue to be removed.

At step 820, portions of the tissue collection device 702 can interact with light 775 from lens 239 to enhance visibility of such portions. As discussed herein, various components of tissue collection device 702 can be configured to reflect light from light source 772 to enhance visibility. For example, tissue separating components, such as teeth 712A and 712B or blade edges, can be reflective or luminescent to enhance display on the video display monitor, such as output unit 18 (FIG. 1).

At step 822, tissue separator 710 of surgical instrument 700 can be used to collect target tissue 754 within the patient. For example, jaws 712AA and 7128B can be navigated through anatomic duct 755 to target tissue 754. Target tissue 754 can comprise tissue that is potentially diseased or otherwise indicative of a diseased condition of the patient. Jaws 712A and 712B can be pushed, pressed or otherwise brought into pressurized contact with target tissue 754. Thus, jaws 712A and 712B can be rotated about hinge 714 from handle section 32 (FIG. 2) to cause jaws 712A and 712B to slice, punch, shave, etc. one or more pieces of tissue away from the anatomy of the patient.

Sample tissue or biological matter separated or collected from the patient at step 822 can be stored within space 756 or internal volume inside tissue collection device 702. For example, separated sample tissue 758 can be positioned within space 756. As explained with reference to biopsy instrument 450 of FIG. 9, biopsy instrument 500 of FIG. 10 and biopsy instrument 550 of FIG. 11, tissue collection device 702 can include capacity enhancing features that facilitate the collection of large volumes of sample tissue, such as multiple sample tissue volumes. Sponge 454 and needle array 456 (FIG. 9) can comprise retention features that can operate independently or cooperatively to bias or retain collected tissue samples within jaws 712A and 712B to prevent dislodging of the collected tissue samples. Slidable rails 510A and 510B (FIG. 10), and deflectable wall 564 (FIG. 11) can comprise capacity increasing features that can be employed to secure increasingly larger volumes of tissue within their respective jaws 712A and 712B.

Additional tissue can be collected with the biopsy device by reapplying tissue separator 710. As more tissue pieces are collected, the newly collected pieces can push the previously collected pieces further into tissue collection device 702. The previously collected pieces can then activate the capacity enhancing features, such as by the previously collected pieces being pushed into engagement with sponge 454, being pushed into needle array 456, pushing movable jaws 504A and 504B outward, and moving deflectable wall 564.

At step 824, tissue collection device 702 can be removed from the patient, such as by removal from the duodenoscope, which can be left in place inside the anatomy. Safeguards can be put into place to ensure removal of the tissue collection device without inadvertently cutting anatomy of the patient. Tissue collection device 702 can be re-inserted if desired to collect additional tissue samples.

At step 826, the collected sample tissue can be removed from tissue collection device 702. For example, jaws 712A and 712B can be rotated away from each other at hinge 714 to access space 756 therebetween and remove sample tissue for analysis, etc.

At step 828, the duodenoscope can be removed from the patient. The patient can thereafter be appropriately closed up or prepared for completion of the procedure.

As such, method 800 illustrates examples of a method of collecting biological matter from internal passages of a patient using activation light integrated into a scope. The activation light can be used to energize light-emitting dye within tissue. The light-emitting dye can be previously administered or metabolized by the patient so that the dye is within the target tissue at the time of the scope procedure. The energizing light from the scope can reach the dyed target tissue by passing through a tissue collection device if conditions warrant. For example, the tissue collection device can be made of material that allows some, most or all light to pass therethrough, while also selectively allowing illumination light to pass through or be reflected back. Thus, the tissue collection device can be optically enhanced (e.g., transparent, clear, reflective, translucent, luminescent, or scattering) to allow the tissue collection device to be at least partially invisible to a camera and recognized by a light source or partially highly visible to a camera, for example. As such, the energizing light can pass through the tissue collection device while the tissue collection device is being employed to collect a sample of target tissue. The present application includes multiple features that can be used to increase the likelihood of successful interventions, such as successfully removing substantially all of diseased tissue or collecting large enough sample sizes that biopsies and other subsequent procedures can be performed on without having to reinsert the forceps or another instrument and without having to perform a subsequent procedure. For example, the present disclosure can combine the ability of collecting large sample sizes (FIGS. 8A-12) and the ability to specificity identify target tissue with a laser and dye combination (FIGS. 13 and 14) to help ensure that a large enough sample of the desirable (properly identified) tissue is collected, thereby reducing or eliminating the need to reinsert an instrument to collect a second sample or perform a follow-on procedure to conduct further tissue collection or additional treatment.

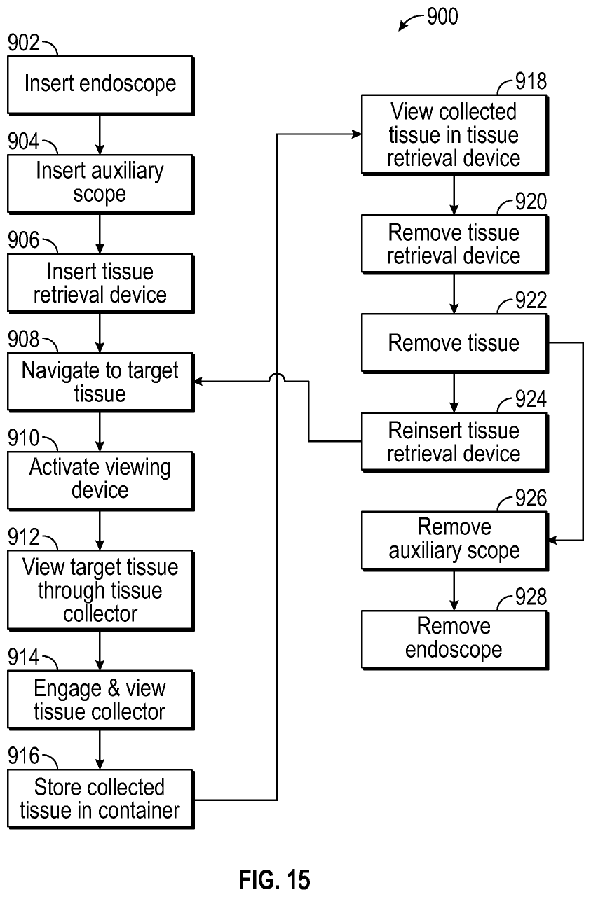

FIG. 15 is block diagram illustrating examples of method 900 of collecting biological matter from a patient using the tissue retrieval devices of the present disclosure, such as those that are optically enhanced. Method 900 can encompass the use of surgical instrument 200 of FIG. 5A-6B and tissue retrieval device 300 of FIG. 7A, as well as other instruments.

At step 902, an endoscope can be inserted into and navigated through anatomy of a patient. For example, endoscope 14 (FIG. 1) can utilize native imaging capabilities to guide insertion section 28 through anatomic ducts of the patient. Insertion section 28 can be bent or curved using control knob 38 to facilitate turning of endoscope 14.

At step 904, an auxiliary scope can be inserted into the endoscope to access anatomy located further in the duct. For example, auxiliary scope 134 (FIG. 4) can be inserted into lumen 62 (FIG. 3C) or lumen 132 (FIG. 4) to reach another anatomic duct intersecting the anatomic duct reached by endoscope 14. Elevator 54 (FIG. 3C) can be used to bend or turn auxiliary scope 134.

At step 906, a tissue retrieval device can be inserted into the auxiliary scope to reach target tissue distal of the auxiliary scope. For example, surgical instrument 200 (FIG. 5A) can be inserted such that tissue collection device 204 extends beyond the distal end of auxiliary scope 134.

At step 908, the tissue collection device can be navigated to the location of target tissue within the patient. For example, tissue collection device 204 can be navigated through an anatomic duct to target tissue 254 (FIG. 6A). The target tissue can comprise tissue that is potentially diseased or otherwise indicative of a diseased condition of the patient.

At step 910, a viewing device on the auxiliary scope can be activated in order to view biological matter of the patient. For example, imaging unit 110 can be activated to view anatomy in field of view 240 of lens 238.

At step 912, target tissue can be viewed using an imaging unit and a video display monitor. For example, imaging unit 110 can use lens 238 to display target tissue on output unit 18. Lens 238 can view the target tissue through transparent or translucent portions of the tissue collection device. Light from a light source can be used to illuminate the target tissue. For example, light from lens 239, as generated by lighting unit 112, can be directed upon the target tissue.

At step 914, a tissue collection device can be pushed, pressed or otherwise brought into pressurized contact with the target tissue. Thus, tissue collection device 204 can be reciprocated axially, or rotated, to cause blade 266 to slice, punch or shave, etc. one or more pieces of tissue away from the anatomy of the patient. Furthermore, portions of the tissue collection device can interact with light from lens 239 to enhance visibility of such portions. For example, tissue separating components, such as teeth or blade edges, can be reflective or luminescent to enhance display on the video display monitor, such as output unit 18.

At step 916, sample tissue or biological matter separated or collected from the patient at step 412 can be stored within a space inside the tissue collection device. For example, as tissue collection device 204 is manipulated back-and-forth, or rotated, separated sample tissue 258 can be positioned within space 256.

At step 918, the retrieved and stored tissue can be viewed within a container of the tissue collection device using an imaging unit and a video display monitor. For example, imaging unit 110 can use lens 238 to display target tissue on output unit 18. Lens 238 can view the stored tissue within transparent or translucent portions of the tissue collection device. Light from a light source can be used to illuminate the target tissue. For example, light from lens 239, as generated by lighting unit 112, can be directed upon the target tissue.

At step 920, the tissue collection device can be removed from the patient, such as by removal from the auxiliary scope, which can be left in place inside the anatomy. Safeguards can be put into place to ensure removal of the tissue collection device without inadvertently cutting anatomy of the patient.

At step 922, the collected sample tissue can be removed from the tissue collection device. For example, jaws 212A and 212B can be rotated away from each other to access space 256 and remove sample tissue 258 for analysis, etc.

Thereafter, method 900 can return to step 908 or can continue to step 920.

At step 924, the tissue collection device can be reinserted. From step 920, steps 908 to 918 can be repeated as many times as desired to achieve a suitable amount of sample tissue, such as a quantity sufficient to perform laboratory testing to ascertain a diagnosis to a high level of certainty. Note that the present disclosure is directed to systems and methods that reduce or eliminate the need to reinsert tissue retrieval devices. However, in some cases it may be desirable to do so in order to collect additional sample material from the same site or to collect sample material from a different site.

At step 926, the auxiliary scope can be removed from the endoscope.

At step 928, the endoscope can be removed from the patient.

As such, method 900 illustrates examples of a method of collecting biological matter from internal passages of a patient in large enough quantities, e.g., by using an optically enhanced (e.g., transparent, clear, reflective, translucent, luminescent, or scattering) tissue removal device with internal storage, to eliminate or reduce insertion and removal of surgical devices from the patient. The optical enhancements allow the tissue removal device to be at least partially invisible to a camera and recognized by a light source, for example.

### Various Notes & Examples

Optically Enhanced Biological Matter Collection Devices for Endoscopes

Example 1 can include or use subject matter such as a tissue separation device comprising an elongate body comprising a proximal end portion and a distal end portion, and a tissue separator coupled to the distal end portion, the tissue separator configured to engage sample tissue for collection, wherein the tissue separator is at least partially made from a material that allows light to pass through the tissue separator.

Example 2 can include, or can optionally be combined with the subject matter of Example 1, to optionally include an endoscope comprising an elongate shaft having a first lumen in which the elongate body can be disposed, and a viewing device configured to view the tissue separator extended from the first lumen by the elongate body.

Example 3 can include, or can optionally be combined with the subject matter of one or any combination of Examples 1 or 2 to optionally include a viewing device is positioned to view through the tissue separator.

Example 4 can include, or can optionally be combined with the subject matter of one or any combination of Examples 1 through 3 to optionally include an endoscope further comprises a light emitter.

Example 5 can include, or can optionally be combined with the subject matter of one or any combination of Examples 1 through 4 to optionally include a tissue separator that comprises forceps.

Example 6 can include, or can optionally be combined with the subject matter of one or any combination of Examples 1 through 5 to optionally include a forceps that comprises a first jaw pivotably connected to the elongate body and a plurality of teeth extending from the first jaw.

Example 7 can include, or can optionally be combined with the subject matter of one or any combination of Examples 1 through 6 to optionally include a plurality of teeth that are fabricated from a material that is florescent.

Example 8 can include, or can optionally be combined with the subject matter of one or any combination of Examples 1 through 7 to optionally include a plurality of teeth that are fabricated from a material that is reflective.

Example 9 can include, or can optionally be combined with the subject matter of one or any combination of Examples 1 through 8 to optionally include a first jaw is that is shaped to form an internal volume.

Example 10 can include, or can optionally be combined with the subject matter of one or any combination of Examples 1 through 9 to optionally include material of the first jaw forming the internal volume is flexible.

Example 11 can include, or can optionally be combined with the subject matter of one or any combination of Examples 1 through 10 to optionally include an opposing jaw disposed opposite the first jaw.

Example 12 can include, or can optionally be combined with the subject matter of one or any combination of Examples 1 through 11 to optionally include a tissue separator that comprises an auger.

Example 13 can include, or can optionally be combined with the subject matter of one or any combination of Examples 1 through 128 to optionally include an auger that comprises a cone-shaped body, a threading extending around the cone-shaped body, and a passage extending into the cone-shaped body. Example 14 can include, or can optionally be combined with the subject matter of one or any combination of Examples 1 through 13 to optionally include threading that is fabricated from a reflective or translucent material.

Example 15 can include, or can optionally be combined with the subject matter of one or any combination of Examples 1 through 14 to optionally include a passage that comprises a sharpened edge.

Example 16 can include, or can optionally be combined with the subject matter of one or any combination of Examples 1 through 15 to optionally include a passage that is fabricated from reflective or translucent material.

Example 17 can include, or can optionally be combined with the subject matter of one or any combination of Examples 1 through 16 to optionally include a tissue separator comprising a punch.

Example 18 can include, or can optionally be combined with the subject matter of one or any combination of Examples 1 through 17 to optionally include a tissue separator that is fabricated from clear polycarbonate or glass.

Example 19 can include, or can optionally be combined with the subject matter of one or any combination of Examples 1 through 18 to optionally include a tissue separator that comprises a tissue removal device having reflective, translucent or transparent properties.

Example 20 can include, or can optionally be combined with the subject matter of one or any combination of Examples 1 through 19 to optionally include a tissue separator comprises an internal volume to store separated tissue.

Example 21 can include or use subject matter such as a method of collecting biological matter using a tissue retrieval device that can comprise inserting the tissue retrieval device into anatomy of a patient, guiding a tissue collector of the tissue retrieval device to a target tissue, viewing the target tissue through the tissue collector, and collecting biological matter from the target tissue with the tissue retrieval device.

Example 22 can include, or can optionally be combined with the subject matter of Example 21, to optionally include viewing the target tissue through the tissue collector by viewing images of the target tissue with a video camera.

Example 23 can include, or can optionally be combined with the subject matter of one or any combination of Examples 21 or 22 to optionally include viewing the target tissue through the tissue collector by viewing target tissue beyond the tissue collector.

Example 24 can include, or can optionally be combined with the subject matter of one or any combination of Examples 21 through 23 to optionally include viewing the target tissue through the tissue collector by viewing target tissue within the tissue collector.

Example 25 can include, or can optionally be combined with the subject matter of one or any combination of Examples 21 through 24 to optionally include penetrating a surface of the target tissue with the tissue collector and viewing newly exposed tissue within the surface that is penetrated.

Example 26 can include, or can optionally be combined with the subject matter of one or any combination of Examples 21 through 25 to optionally include collecting biological matter from the target tissue with the tissue retrieval device by operating forceps to sever a portion of the target tissue.

Example 27 can include, or can optionally be combined with the subject matter of one or any combination of Examples 21 through 26 to optionally include collecting biological matter from the target tissue with the tissue retrieval device by boring a tissue punch into the target tissue.

Example 28 can include, or can optionally be combined with the subject matter of one or any combination of Examples 21 through 27 to optionally include collecting biological matter from the target tissue with the tissue retrieval device by advancing an auger into the target tissue.

Example 29 can include, or can optionally be combined with the subject matter of one or any combination of Examples 21 through 283 to optionally include illuminating the tissue retrieval device with a light emitter.

Example 30 can include, or can optionally be combined with the subject matter of one or any combination of Examples 21 through 29 to optionally include fluorescing a cutting edge of the tissue retrieval device.

Example 31 can include, or can optionally be combined with the subject matter of one or any combination of Examples 21 through 29 to optionally include reflecting light with a cutting edge of the tissue retrieval device.

Example 32 can include, or can optionally be combined with the subject matter of one or any combination of Examples 21 through 31 to optionally include flexing the tissue retrieval device to allow a cutting edge of the tissue retrieval device to engage tissue.

Example 33 can include, or can optionally be combined with the subject matter of one or any combination of Examples 21 through 32 to optionally include storing tissue within the tissue retrieval device.

Example 34 can include, or can optionally be combined with the subject matter of one or any combination of Examples 21 through 23 to optionally include viewing the target tissue through the tissue collector by viewing the target tissue through translucent material of the tissue collector.

Example 35 can include, or can optionally be combined with the subject matter of one or any combination of Examples 21 through 34 to optionally include inserting an endoscope comprising a first longitudinal passage into the anatomy of the patient, inserting an auxiliary scope comprising a second longitudinal passage into the first longitudinal passage, inserting the tissue retrieval device into the second longitudinal passage to reach the target tissue.

Example 36 can include or use subject matter such as a surgical instrument that can comprise an endoscope comprising an insertion shaft extending from a proximal end to a distal end, a working channel extending at least partially through the insertion shaft, and an imaging system coupled to the insertion shaft, the imaging system having a field of view projecting distally of the working channel, a tissue retrieval device comprising an elongate shaft positionable in the working channel, the elongate shaft extending along an axis and configured for insertion into anatomy, and a tissue collection device coupled to the elongate shaft and configured to separate tissue from the anatomy, wherein the tissue collection device is optically enhanced to interact with the field of view of the imaging system.

Example 37 can include, or can optionally be combined with the subject matter of Example 36, to optionally include a tissue collection device that is configured to be invisible to the imaging system.

Example 38 can include, or can optionally be combined with the subject matter of one or any combination of Examples 36 or 37 to optionally include a tissue collection device that is fabricated of a translucent material.

Example 39 can include, or can optionally be combined with the subject matter of one or any combination of Examples 36 through 38 to optionally include a tissue collection device that is fabricated of a transparent material.

Example 40 can include, or can optionally be combined with the subject matter of one or any combination of Examples 36 through 39 to optionally include a tissue collection device that is configured to magnify images obtained by the imaging system.

Example 41 can include, or can optionally be combined with the subject matter of one or any combination of Examples 36 through 40 to optionally include a tissue collection device that comprises a tissue separator device that is configured to be highlighted by the imaging system.

Example 42 can include, or can optionally be combined with the subject matter of one or any combination of Examples 36 through 41 to optionally include a tissue separator device is fabricated of a reflective material.

Example 43 can include, or can optionally be combined with the subject matter of one or any combination of Examples 36 through 42 to optionally include a tissue collection device is fabricated of a translucent material.

Tethered Biological Matter Collection Devices for Endoscopes

Example 1 is a biopsy instrument comprising: a tissue separator device comprising: a base; a tissue separator mounted to the base; and an actuation mechanism coupled to the tissue separator; and an elongate control element coupled to the actuation mechanism to manipulate the tissue separator; wherein the tissue separator is at least partially made of a material capable of transmitting light.

In Example 2, the subject matter of Example 1 optionally includes an endoscope comprising: an elongate shaft having a first lumen in which the elongate control element can be disposed; and a viewing device configured to view the tissue separator device distal of the first lumen by the elongate body.

In Example 3, the subject matter of Example 2 optionally includes wherein: the first lumen extends along a longitudinal axis and has a first height extending transverse to the longitudinal axis; and the tissue separator has a second height that is larger than the first height.

In Example 4, the subject matter of any one or more of Examples 2-3 optionally include wherein the base has a third height that is larger than the first height.

In Example 5, the subject matter of any one or more of Examples 2-4 optionally include wherein the base is couplable to the elongate shaft.

In Example 6, the subject matter of any one or more of Examples 2-5 optionally include wherein the elongate control element is longer than the endoscope lumen.

In Example 7, the subject matter of any one or more of Examples 1-6 optionally include a quick connect coupling at a proximal end of the elongate control member.

In Example 8, the subject matter of Example 7 optionally includes a handpiece couplable to the quick connect coupling, the handpiece configured to activate the actuation mechanism via the elongate control element.

In Example 9, the subject matter of any one or more of Examples 1-8 optionally include wherein the tissue separator comprises forceps comprising: a first jaw; a second jaw; and a hinge about which at least one of the first and second jaws is configured to articulate between open and closed positions.

In Example 10, the subject matter of Example 9 optionally includes wherein the actuation mechanism comprises a lever extending from the at least one of the first and second jaws configured to articulate between the open and closed positions.

In Example 11, the subject matter of any one or more of Examples 9-10 optionally include wherein the forceps include a capacity enhancement feature configured to allow the forceps to hold multiple tissue samples.

In Example 12, the subject matter of Example 11 optionally includes wherein the capacity enhancement feature comprises a tissue biasing device configured to immobilize a piece of tissue retained between the first and second jaws in the open position.

In Example 13, the subject matter of Example 12 optionally includes wherein the tissue biasing device comprises: a sponge located on the first jaw; and a needle array located on the second jaw positioned to penetrate the sponge when the first and second jaws are in the closed position.

In Example 14, the subject matter of any one or more of Examples 12-13 optionally include wherein the capacity enhancement feature comprises a capacity increasing feature configured to increase an internal volume of at least one of the first and second jaws.

In Example 15, the subject matter of Example 14 optionally includes wherein the capacity increasing feature comprises at least one of the first jaw and the second jaw being configured to translate in a direction opposite the other of the first and second jaws at the hinge to increase the space between the first and second jaws.

In Example 16, the subject matter of any one or more of Examples 14-15 optionally include wherein the capacity increasing feature comprises at least one of the first jaw and the second jaw being configured with a flexible wall to increase the space between the first and second jaws.

In Example 17, the subject matter of any one or more of Examples 9-16 optionally include wherein at least one of the first jaw and the second jaw is fabricated at least partially from transparent material.

In Example 18, the subject matter of any one or more of Examples 1-17 optionally include wherein the tissue separator comprises an auger comprising: a cone-shaped body; a threading extending around the cone-shaped body; and a passage extending into the cone-shaped body.

In Example 19, the subject matter of any one or more of Examples 1-18 optionally include wherein the elongate control element comprises a flexible pull wire.

In Example 20, the subject matter of any one or more of Examples 1-19 optionally include a biasing element for the actuation mechanism to bias the tissue separator to a predetermined position.

Example 21 is a method of collecting biological matter using a biopsy instrument, the method comprising: tethering the biopsy instrument to a distal end portion of an endoscope; inserting the endoscope with the biopsy instrument into anatomy of a patient; guiding the biopsy instrument to a target tissue site; viewing target tissue through the biopsy instrument; and collecting biological matter from the target tissue site with the biopsy instrument.

In Example 22, the subject matter of Example 21 optionally includes wherein guiding the biopsy instrument to the target tissue site comprises viewing the anatomy through the biopsy instrument.

In Example 23, the subject matter of any one or more of Examples 21-22 optionally include wherein viewing the target tissue through the biopsy instrument comprises viewing images of the target tissue site with a video camera.

In Example 24, the subject matter of any one or more of Examples 21-23 optionally include wherein tethering the biopsy instrument to the distal end portion of the endoscope comprises: inserting an elongate control member into a lumen of the endoscope from the distal end of the endoscope.

In Example 25, the subject matter of Example 24 optionally includes wherein tethering the biopsy instrument to the distal end portion of the endoscope comprises engaging a base of the biopsy instrument to a distal end of the endoscope.

In Example 26, the subject matter of any one or more of Examples 24-25 optionally include attaching a control device to the elongate control member at a proximal end portion of the endoscope.

In Example 27, the subject matter of any one or more of Examples 21-26 optionally include wherein collecting biological matter from the target tissue with the biopsy instrument comprises operating forceps to sever a portion of the target tissue.

In Example 28, the subject matter of Example 27 optionally includes wherein collecting biological matter from the target tissue with the biopsy instrument comprises retaining biological matter within the forceps with a tissue biasing device.

In Example 29, the subject matter of Example 28 optionally includes wherein retaining biological matter within the forceps with the tissue biasing device further comprises piercing the collected tissue with a tine.

In Example 30, the subject matter of any one or more of Examples 27-29 optionally include wherein collecting biological matter from the target tissue with the biopsy instrument comprises increasing a capacity of the forceps with a capacity increasing feature.

In Example 31, the subject matter of Example 30 optionally includes wherein increasing the capacity of the forceps with the capacity increasing feature comprises sliding a jaw of the forceps away from a pivot point to increase a distance from an opposing jaw.

In Example 32, the subject matter of any one or more of Examples 30-31 optionally include wherein increasing the capacity of the forceps with the capacity increasing feature comprises flexing a wall of a jaw of the forceps to increase an internal volume of the jaw.

In Example 33, the subject matter of any one or more of Examples 21-32 optionally include obtaining multiple tissue samples from the target tissue with the biopsy instrument before withdrawing the endoscope from the anatomy.

Translucent Biopsy Device with Laser Fluorescing Capabilities

Example 1 is a surgical instrument comprising: a scope comprising: an elongate body extending from a proximal end portion to a distal end portion; a working channel extending at least partially through the elongate body; an imaging component coupled to the elongate body, the imaging component having a field of view projecting distally of the working channel; and a first light emitter configured to project light of a first wavelength from the elongate body suitable for energizing a light-emitting material; and a tissue retrieval device comprising: an elongate shaft positionable in the working channel, the elongate shaft extending along an axis and configured for insertion into anatomy; and a tissue collection device coupled to the elongate shaft and configured to separate tissue from the anatomy.

In Example 2, the subject matter of Example 1 optionally includes the imaging component being positioned to view through the tissue collection device.

In Example 3, the subject matter of any one or more of Examples 1-2 optionally includes the first light emitter being configured to fluoresce a fluorophore.

In Example 4, the subject matter of any one or more of Examples 1-3 optionally includes the first light emitter comprising a laser.

In Example 5, the subject matter of any one or more of Examples 1-4 optionally includes the first wavelength comprising 358 to 405 nanometers.

In Example 6, the subject matter of any one or more of Examples 1-5 optionally includes the scope further comprises a second light emitter configured to project light of a second wavelength from the elongate body.

In Example 7, the subject matter of Example 6 optionally includes the second light emitter comprising a light emitting diode.

In Example 8, the subject matter of any one or more of Examples 6-7 optionally includes the second wavelength comprising 400 to 700 nanometers.

In Example 9, the subject matter of any one or more of Examples 1-8 optionally includes a light transmitting fiber extending through the elongate body to the first light emitter.

In Example 10, the subject matter of Example 9 optionally includes the light transmitting fiber being embedded in material of the elongate body.

In Example 11, the subject matter of any one or more of Examples 9-10 optionally includes the light transmitting fiber extending at least partially through a lumen in the elongate body.

In Example 12, the subject matter of any one or more of Examples 1-11 optionally includes a light source connected to the first light emitter.

In Example 13, the subject matter of Example 12 optionally includes the light source being carried by the scope.

In Example 14, the subject matter of any one or more of Examples 1-13 optionally include the tissue collection device comprising forceps.

In Example 15, the subject matter of Example 14 optionally includes the forceps comprising: a first jaw pivotably connected to the elongate body; and a plurality of teeth extending from the first jaw.

In Example 16, the subject matter of Example 15 optionally includes the plurality of teeth being fabricated from a material that is florescent.

In Example 17, the subject matter of any one or more of Examples 15-16 optionally includes the plurality of teeth being fabricated from a material that is reflective.

In Example 18, the subject matter of any one or more of Examples 1-17 optionally includes the tissue collection device comprises an internal volume to store separated tissue.

In Example 19, the subject matter of any one or more of Examples 1-18 optionally includes the tissue collection device being fabricated from clear polycarbonate or glass.

In Example 20, the subject matter of any one or more of Examples 1-19 optionally includes the tissue collection device being optically enhanced to interact with the field of view of the imaging component, the optical enhancement comprises reflective, translucent, transparent or magnification properties.

Example 21 is a method of collecting biological matter using a tissue retrieval device, the method comprising: inserting the tissue retrieval device into anatomy of a patient; guiding a tissue collector of the tissue retrieval device to a target tissue; illuminating the target tissue with an energization light; viewing light-emitting target tissue through the tissue collector; and collecting biological matter from the target tissue with the tissue collector.

In Example 22, the subject matter of Example 21 optionally includes viewing light-emitting target tissue through the tissue collector.

In Example 23, the subject matter of Example 22 optionally includes viewing the target tissue through the tissue collector by viewing images of the target tissue with a camera beyond the tissue collector.

In Example 24, the subject matter of Example 23 optionally includes wherein viewing images of the target tissue with the camera beyond the tissue collector comprises viewing light emitted from the target tissue.

In Example 25, the subject matter of any one or more of Examples 21-24 optionally include penetrating a surface of the target tissue with the tissue collector and viewing newly exposed tissue within the surface that is penetrated.

In Example 26, the subject matter of any one or more of Examples 21-25 optionally include illuminating the target tissue with the energization light by illuminating the target tissue with a laser.

In Example 27, the subject matter of any one or more of Examples 21-26 optionally include illuminating the target tissue with the energization light to cause the target tissue to emit light.

In Example 28, the subject matter of any one or more of Examples 21-27 optionally include illuminating the target tissue with an energization light by fluorescing the target tissue; and viewing light-emitting target tissue comprises viewing fluoresced target tissue.

In Example 29, the subject matter of any one or more of Examples 21-28 optionally include infusing a fluorescent material into the target tissue to produce the light-emitting target tissue.

In Example 30, the subject matter of any one or more of Examples 21-29 optionally include illuminating the tissue retrieval device with a second light emitter.

In Example 31, the subject matter of Example 30 optionally includes the second light emitter emitting visible light.

In Example 32, the subject matter of Example 31 optionally includes the energization light having a wavelength in the range of 358 to 405 nanometers; and the visible light having a wavelength in the range of 400 to 700 nanometers.

In Example 33, the subject matter of any one or more of Examples 21-32 optionally include fluorescing a cutting edge of the tissue collector.

In Example 34, the subject matter of any one or more of Examples 21-33 optionally include reflecting light with a cutting edge of the tissue collector.

In Example 35, the subject matter of any one or more of Examples 21-34 optionally includes viewing the target tissue through the tissue collector by viewing the target tissue through translucent material of the tissue collector.

In Example 36, the subject matter of any one or more of Examples 21-35 optionally include inserting an endoscope comprising a first longitudinal passage into the anatomy of the patient; inserting an auxiliary scope comprising a second longitudinal passage into the first longitudinal passage; and inserting the tissue retrieval device into the second longitudinal passage to reach the target tissue.

In Example 37, the subject matter of any one or more of Examples 21-36 optionally includes collecting biological matter from the target tissue with the tissue collector by operating forceps to sever a portion of the target tissue.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A surgical instrument comprising:
an elongate body comprising:
a proximal end portion;
a distal end portion; and
a bendable shaft extending from the proximal end portion to the distal end portion; and
a tissue separator coupled to the distal end portion, the tissue separator configured to engage sample tissue for collection;
wherein the tissue separator is at least partially made from a material that allows light to pass through the tissue separator.

2. An endoscopy system comprising the surgical instrument of claim 1, further comprising an endoscope comprising:
an elongate bendable shaft having a first lumen in which the elongate body can be disposed; and
a viewing device configured to view the tissue separator extended from the first lumen by the elongate body;
wherein the viewing device is positioned to view through the tissue separator.

3. The surgical instrument of claim 1, wherein the tissue separator comprises a tissue removal device having reflective, translucent, transparent or magnification properties.

4. The surgical instrument of claim 1, wherein the tissue separator is fabricated from clear polycarbonate or glass.

5. The surgical instrument of claim 1, wherein the tissue separator comprises an internal volume to store separated tissue.

6. The surgical instrument of claim 1, wherein the tissue separator comprises an auger or a punch.

7. The surgical instrument of claim 1, wherein the tissue separator comprises forceps comprising:
a first jaw;
a plurality of teeth extending from the first jaw; and
a second jaw disposed opposite the first jaw;
wherein the first jaw is pivotable from a closed position engaged with the second jaw to an open position away from the second jaw; and
wherein an internal storage volume is defined between the first jaw and the second jaw.

8. The surgical instrument of claim 7, wherein the plurality of teeth are fabricated from a material that is florescent or reflective.

9. The surgical instrument of claim 7, wherein the forceps include a capacity enhancement feature configured to allow the forceps to hold multiple tissue samples.

10. The surgical instrument of claim 9, wherein the capacity enhancement feature comprises a tissue biasing device configured to immobilize a piece of tissue retained between the first and second jaws in the open position.

11. The surgical instrument of claim 10, wherein the tissue biasing device comprises:
a sponge located on the first jaw; and
a needle array located on the second jaw positioned to penetrate the sponge when the first and second jaws are in the closed position.

12. The surgical instrument of claim 9, wherein the capacity enhancement feature comprises a capacity increasing feature configured to increase an internal volume of at least one of the first and second jaws.

13. The surgical instrument of claim 12, wherein the capacity increasing feature comprises at least one of the first jaw and the second jaw being configured to translate in a direction opposite the other of the first and second jaws at a hinge to increase the internal storage volume between the first and second jaws.

14. The surgical instrument of claim 12, wherein the capacity increasing feature comprises at least one of the first jaw and the second jaw being configured with a flexible wall to increase the internal storage volume between the first and second jaws.

15. The surgical instrument of claim 1, further comprising a first light emitter connected to the elongate body and configured to project light distal to the distal end portion at a first wavelength.

16. The surgical instrument of claim 15, wherein the first light emitter is configured to fluoresce a fluorophore.

17. The surgical instrument of claim 15, wherein the first light emitter comprises a laser and the first wavelength comprises 358 to 405 nanometers.

18. The surgical instrument of claim 15, further comprising a second light emitter connected to the elongate body and configured to project light distal to the distal end portion at a second wavelength different from the first wavelength.

19. The surgical instrument of claim 18, wherein the second light emitter comprises a light emitting diode and the second wavelength comprises 400 to 700 nanometers.

20. The surgical instrument of claim 15, further comprising a light transmitting fiber extending through the elongate body to the first light emitter.

21. A method of collecting biological matter using a tissue retrieval device, the method comprising:
inserting the tissue retrieval device into anatomy of a patient;
guiding a tissue collector of the tissue retrieval device to a target tissue;
viewing the target tissue through the tissue collector; and
collecting biological matter from the target tissue with the tissue retrieval device.

22.-40. (canceled)
